(19) United States
(12) Patent Application Publication
(10) Pub. No.: US 2010/0129398 A1
(43) Pub. Date: May 27, 2010

(54) Materials and Methods for Control of Porcine Reproductive and Respiratory Syndrome

(75) Inventors: Kelly L. Klinge, Ames, IA (US); Michael B. Roof, Ames, IA (US)

Correspondence Address: Michael P. Morris;Boehringer Ingelheim USA Corporation, 900 Ridgebury Road, Ridgefield, CT, 06877-0368, US

(73) Assignee: BOEHRINGER INGELHEIM VETMEDICA, INC., Saint Joseph, MO (US)

(21) Appl. No.: 12/620,495

(22) Filed: Nov. 17, 2009

Related U.S. Application Data
(60) Provisional application No. 61/118,408, filed on Nov. 26, 2008.

(51) Int. Cl. A61K 39/12; A61P 31/14

(52) U.S. Cl. 424/204.1

(57) ABSTRACT

Methods of reducing the severity of porcine reproductive and respiratory syndrome virus (PRRSV) infections, as well as, methods of preventing such infections are provided. The methods provide for the age-based innoculation of swine with PRRS antigen, preferably Ingelvac® ATP.

### FIELD OF THE INVENTION

The present invention relates to methods for control of porcine reproductive and respiratory syndrome (PRRS). Immunogenic compositions and methods of using them to reduce the incidence or severity of porcine reproductive and respiratory syndrome infection are described.

### BACKGROUND OF THE INVENTION

Porcine reproductive and respiratory syndrome (PRRS) is viewed by many as the most important disease currently affecting the pig industry worldwide. The syndrome first was described in 1987 in the United States as “mystery swine disease” and rapidly spread across the globe. It causes severe reproduction losses, is associated with increased mortality due to secondary infections, and is linked to reduced feed conversion and average daily weight gain. Unfortunately, control of the virus that causes PRRS has proven to be difficult.

Transmission of the PRRS virus (PRRSV) can, and often does, occur through direct contact between infected and susceptible pigs. Transmission over very short distances by air or through semen also may occur. Once infected, the virus can remain in the blood of adults for about two weeks, and in infected pigs for one to two months or more. Infected boars may shed the virus in the semen for more than 100 days. This long period of viremia significantly increases the possibility of transmission. In addition, the PRRS virus can cross the placenta during the last third of the gestation period to infect piglets in utero and cause stillbirth or weak-born piglets.

All types and sizes of herds, including those with high or ordinary health status or from either indoor or outdoor units, can be infected with PRRS virus. Infected herds may experience severe reproductivity losses, as well as, increased levels of post weaning pneumonia with poor growth. The reproductive phase typically lasts for two to three months; however, post weaning problems often become endemic. The reproductive disease is characterized by an abortion outbreak that affects both sows and gilts in the last term of gestation. Premature farrowings around 109 and 112 days of gestation occur. The number of stillbirths and weak-born piglets increases and results in a considerable increase in pre-weaning mortality.

The respiratory phase traditionally has been seen in the nursery, especially in continuous flow nurseries. However, respiratory problems caused by PRRS virus can also be seen in the finisher as part of the porcine respiratory disease complex (PRDC). A reduction in growth rate, an increase in the percentage of unmarketable pigs, and elevated post weaning mortality can occur. Diagnostic findings indicate high levels of pneumonia that associate with the PRRS virus together with a wide variety of other microbials commonly seen as secondary infectious agents. Bacterial isolates may include Streptococcus suis, Haemophilus suis, Actinobacillus pleuropneumoniae, Actinobacillus suis, Mycoplasma hyopneumoniae, and Pasteurella multocida among others. Viral agents commonly involved include swine influenza virus and porcine respiratory corona virus. Affected pigs rarely respond to high levels of medication, and all-in/all-out systems have failed to control the disease.

Pigs recovered from a PRRS infection will develop an immune response, which under normal circumstances will protect them from being infected again by the same virus strain. However, PRRS virus has the ability to change (by mutation or recombination); and therefore, new viral strains may arise. In such cases, cross protection between strains may not exist, and new outbreaks may be observed in farms that had been infected previously.

Age- and viral strain-dependent variation in porcine responses to PRRSV infection was previously reported. However, the significance of the findings is uncertain since mature adult pigs were not included, quantitative viral loads were not determined, the viruses were extremely different in genetics as well as virulence, and there was coincident disease in the control group.

Better treatments or vaccines that can reduce the severity of disease, reduce infectivity, or prevent PRRSV are needed.

### SUMMARY OF THE INVENTION

The invention provides methods of treating or reducing the severity of porcine reproductive and respiratory syndrome virus (PRRSV) infection, as well as, methods of preventing PRRSV infection.

Generally, the method is for treating or reducing the severity of or incidence of porcine reproductive and respiratory syndrome virus (PRRSV) infection. “Treating or reducing the severity of or incidence of” refers to a reduction in the severity of clinical signs, symptoms, and/or pathological signs normally associated with infection, up to and including prevention of any such signs or symptoms. “Pathological signs” refers to evidence of infection that is found microscopically or during necropsy (e.g. lung lesions).

The method generally includes the step of administering a therapeutic amount of a PRRSV antigen to a swine of a defined age or age range. For example, in one aspect of the invention, one therapeutic amount of a PRRSV antigen may be administered to a piglet about three-weeks-old or younger, and different therapeutic amounts of the antigen may be administered to a pig between about 3 weeks of age and 4 weeks of age. Similarly, an even different therapeutic amount might be administered to a pig between about four weeks and sixteen weeks of age (or any age within this range, e.g. five weeks to six weeks of age, nine weeks to fifteen weeks of age, seven weeks to ten weeks of age, etc), or to pig older than sixteen weeks, such as an adult sow.

Preferably the PRRSV antigen is a modified live PRRS virus and more preferably the PRRSV antigen is Ingelvac® ATP. The PRRSV antigen can be administered in any conventional fashion and in the case of Ingelvac® ATP, the preferred method of administration is nasally. It is preferred that the administered PRRSV antigen provide its benefits of treating or reducing the severity of or incidence of PRRSV infection after a single dose, as with Ingelvac® ATP, however, if other antigens are selected, they will be administered in their conventional fashion, which may include one or more booster doses after the initial administration. Those of skill in the art will be able to determine appropriate dosing levels based on the PRRSV antigen selected and the age range of the animal to which the antigen will be administered.

In one aspect of the invention, a particular dose regimen is selected based on the age of the pig and antigen selected for administration. This will permit pigs of any age to receive the most efficacious dose based on the present invention's discovery that PRRSV infection (from both wild type exposure and vaccination) is cleared much more quickly in older animals. Thus, in some respects, vaccination of older animals is preferred but that vaccination of younger pigs, including those three weeks of age and younger helps to induce active immunity and is still very beneficial as having higher viral titers in three week old pigs may induce better immunity. As shown herein, animal age is a critical factor in PRRS control and may be a factor that impacts vaccination and development of an effective immune response. Thus, age, innate, and active immunity are important and need to be considered in control strategies.

In a preferred method, a therapeutic amount of Ingelvac® ATP is administered to a pig or piglet that is about three weeks old. The amount selected will vary depending upon the age of the pig. Alternatively, a different therapeutic amount of Ingelvac® ATP is administered to a pig or piglet that is older than about 3 weeks, and this amount will also change as the pig receiving such an administration ages or becomes older. Accordingly, pigs about four weeks old, six weeks old, eight weeks old, ten weeks old, twelve weeks old, fourteen weeks old, sixteen weeks old, a gilt, or a sow will all receive different amounts. Preferably, the Ingelvac® ATP is administered nasally; however, other methods of administration such as intramuscular, dermal, retinal, oral, subcutaneous, and the like, that are well-known and used in the art may be used.

A preferred therapeutic dose of Ingelvac® ATP is about two milliliters (2 mLs). Skilled artisans will recognize that the dosage amount may be varied based on the breed, size, and other physical factors of the individual subject, as well as, the specific formulation of Ingelvac® ATP and the route of administration.

Preferably, the Ingelvac® ATP is administered in a single dose; however, additional doses may be useful. Again, the skilled artisan will recognize through the present invention that the dosage and number of doses is influenced by the age and physical condition of the subject pig, as well as, other considerations common to the industry and the specific conditions under which the Ingelvac® ATP is administered.

In another aspect of the present invention a method of determining the proper timing and dosage for vaccination of a pig against PRRSV is provided. The method generally comprises the steps of determining at least one variable selected from the group consisting of age, health status, innate immunity level and active immunity level, of the pig, and adjusting a standard dosage level to account for these variables. Generally, the innate immunity level and active immunity level will be determined by referring to a standard comprised of average levels from a population of pigs of similar age and health status. In a particularly preferred method, all variable are considered prior to determining the optimum dosage level and timing of administration.

In one aspect of the present invention, provided herein is a method of detecting a virulent PRRSV infection in piglets comprising the step of periodically obtaining a blood sample for the piglet and monitoring the levels of IL-10 in blood serum of said piglet, wherein an increase in IL-10 concentration up to 40 pg/mL indicates a virulent PRRSV infection.

In another aspect of the present invention, provided herein is a method of differentiating between viral persistence and viral pathogenesis in pigs, comprising the steps of determining the age of the pig; obtaining a blood serum sample from the pig and determining the serum concentration of IL-10 in the blood serum, wherein if the pig is a less than 8 weeks old, the presence of IL-10 concentration up to 40 pg/mL indicates virulent pathogenesis and not persistent viremia.

In one aspect of the present invention, provided herein is a method of gauging the effect of anti-viral treatment in piglets or screening an anti-viral compositions comprising the steps of administering a candidate composition to the piglet and monitoring the level of IL-10 in the blood serum of the piglet, wherein the composition capable reducing IL-10 levels to the lowest level in the shortest treatment period is the most effective composition.

Unless defined otherwise, all technical and scientific terms used herein have the same meaning as is commonly understood by one of skill in the art to which this invention belongs at the time of filing. All patents and publications referred to herein are incorporated by reference herein.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows the Group Mean TCID₅₀ (Tissue Culture Infectious Doses) results and is a compilation of the data presented separately in FIGS. 2-7.

FIG. 2 illustrates the ATP Group Mean TCID₅₀ results.

FIG. 3 shows the JA142 Group Mean TCID₅₀ results.

FIG. 4 illustrates the Strict Controls Group Mean TCID₅₀ results.

FIG. 5 shows the 3 Week Piglet Group Mean TCID₅₀ results.

FIG. 6 illustrates the 16 Week Pig Group Mean TCID₅₀ results.

FIG. 7 shows the Sow Group Mean TCID₅₀ results.

FIG. 8 illustrates a compilation of the Group Mean Quantitative PCR (qPCR) results that are presented separately in FIGS. 9-14.

FIG. 9 shows the ATP Group Mean Quantitative PCR Results.

FIG. 10 shows the JA142 Group Mean Quantitative PCR Results.

FIG. 11 illustrates the Strict Control Group Mean Quantitative PCR Results.

FIG. 12 shows the Three Week Piglet Group Mean Quantitative PCR Results.

FIG. 13 illustrates the Sixteen Week Pig Group Mean Quantitative PCR Results.

FIG. 14 shows the Sow Group Mean Quantitative PCR Results.

FIG. 15 shows the Group Mean IDEXX PRRS ELISA results and is compilation of the data presented in FIGS. 16-21.

FIG. 16 illustrates the ATP Group Mean IDEXX PRRS ELISA results.

FIG. 17 shows the JA142 Group Mean IDEXX PRRS ELISA results.

FIG. 18 shows the Strict Controls Group Mean IDEXX PRRS ELISA results.

FIG. 19 illustrates the Three Week Piglets Group Mean IDEXX PRRS ELISA Results.

FIG. 20 shows the Sixteen Week Pig Group Mean IDEXX PRRS ELISA Results.

FIG. 21 illustrates the Sow Group mean IDEXX PRRS ELISA Results.

FIG. 22 illustrates the Group Mean IDEXX M. hyo ELISA results. In all cases at day 28 the sixteen week old pigs yields the highest S/P ratios, and the three week old pigs had the lowest S/P ratios. Sixteen week old pigs treated with JA142 or ATP had similar S/P ratios at day 28 but the S/P ratio dropped at day 63 for those treated with JA142. Sows treated with ATP had a higher S/P ratio at both days 28 and 63 than sows treated with JA142. Three week old pigs treated with JA142 had a higher S/P ratio than three week old pigs treated with ATP at both days 28 and 63.

FIG. 23 shows the Group Mean Gross Lung Lesion Scores at Day 63.

FIG. 24 provides the Group Mean Average Daily Weight Gain in pounds.

FIG. 25 illustrates the Group Mean PRRS Immunohistochemistry Scores.

FIG. 26 shows the Group Mean Clinical Scores.

FIG. 27 shows the effect of age and viral strain on antibody responses to specific structural and nonstructural PRRSV proteins. (A) Anti-nucleocapsid antibodies. (B) Anti-nonstructural protein 2 antibodies. Data are ELISA absorbance values (mean±1 standard deviation) of 10 animals per group. Treatment group legend is shown in panel A.

FIG. 28 shows the effect of pig age and PRRSV strain on interferon (IFN) γ-secreting cell frequencies in peripheral blood mononuclear cells (PBMC). Panels A-C, control uninfected pigs (4-5 pigs per group); D-F, pigs inoculated with attenuated ATP PRRSV (5-8 pigs per group); G-I, pigs infected with virulent JA142 (10-11 pigs per group). Panels A, D, and G, PBMC cultured without stimulation; B, E, and H, PBMC cultured with JA142 PRRSV; C, F, and I; PBMC cultured in presence of phytohemagluttinin (PHA). In each panel, piglets are open squares, finisher pigs are open circles, and sows are closed circles. Asterisk in panels F and I indicate wells with too many cells to count, i.e. >400 per well.

FIG. 29 shows the effect of pig age and viral strain on IL-10 levels in serum early in infection. Data points are values from individual piglets (A), finishers (B), and sows (C) treated as indicated in the legend (box in panel A).

### DETAILED DESCRIPTION

The purpose of this study was to evaluate the impact of age and immune response on PRRSV in vivo replication, persistence, and its ability to cause disease. Immune responses were elicited and evaluated among three different age groups, each of which received three different treatments of varying virulence. Results of the study yielded new methods and compositions for treating a PRRSV infection that relate to the age at which innoculation against or treatment of PRRS infection occurs.

In one aspect of the invention, animal age, likely due to increased innate immune resistance, strongly influences the outcome of acute PRRSV infection, whereas an effective antibody response is triggered at a low threshold of infection that is independent of age. Prolonged infection is not due to IL-10-mediated immunosuppression, and PRRSV does not elicit a specific IFN γ response, especially in non-adult animals. Equivalent antibody responses are elicited in response to virulent and attenuated viruses, indicating that the antigenic mass necessary for an immune response is produced at a low level of infection, and is not predicted by viremic status. Thus, viral replication occurs in lung or lymphoid tissues even though viremia is not always observed.

A total of ninety pigs from the same PRRSV-free genetic source, comprising thirty pigs in each of three different age groups: three week old weaned piglets (herein referred to as three week old or 3 week piglets), growing pigs at 16-20 weeks of age (herein referred to as sixteen week old or 16 week pigs), and mature, non-bred sows at 3±1 parity. On day 0, ten pigs from each age group received one of the following treatments: JA142 virulent PRRSV (parental isolate of vaccine), avirulent PRRSV (Ingelvac® PRRS ATP), or placebo. Viremia and the humoral response in the PRRSV-exposed animals were monitored for 63 days. The data collected illustrate distinct trends among the age groups.

The TCID₅₀ evaluation of viremia following PRRSV exposure revealed that the three week old piglets generated the highest virus titers and maintained the live virus in the bloodstream the longest. A modest statistical difference was observed between sixteen week pigs and sows. These observations were confirmed by quantitative RT-PCR and ELISA analyses. Thus, age of exposure was found to significantly effect the viremic and immunological outcomes.

Weaned three week old piglets demonstrated higher viral genome titers and longer persistence in the blood than older animals. And, older animals, both sows and pigs sixteen to twenty weeks old, seroconverted sooner and achieved higher group average S/P ratios than the piglets.

Viral PRRS isolates of different virulence levels illicit distinct responses in the host. The parent virus (JA142) tended to induce detectable levels of virus in the sera about two days prior to that of the attenuated culture (Ingelvac® ATP). The JA142 virus generally replicated to higher overall titers (˜2.0 logs via TCID₅₀) in the sera as well as induced earlier seroconversion via PRRS IDEXX ELISA by about two or so days.

The TCID₅₀ data show a distinct delay in viremia in all ages of pigs receiving the Ingelvac® PRRS ATP when compared to those pigs receiving the virulent JA142 challenge strain. These results further illustrate the known difference in virulence between the vaccine and its virulent parent strain. Further, a noticeable trend developed among pig ages within each treatment group. The youngest pigs achieved higher levels of viremia and retained those higher levels longer than both of the older groups of pigs.

Upon statistical analysis, the differences in levels of viremia over time when comparing data from all treatment groups are significant (p≦0.05). Also, the differences within the ATP and the JA142 treatments are significant among all age groups.

By varying the age of the host at the time of infection, additional trends were revealed. The three week old piglets (three week piglets, 3 week piglets) displayed about 40 days of viremia via TCID₅₀; whereas, the sixteen week old pigs (sixteen week pigs, 16 week pigs) and the sows displayed only two weeks of viremia. Regardless of the virus used as challenge, the three week piglets demonstrated about 2.0 logs higher levels of in vivo replication than the sixteen week pigs and the sows. Seroconversion tested via the PRRS IDEXX ELISA told a different story. The sows achieved higher overall S/P ratios as well as demonstrating increases in S/P ratio earlier than the younger groups of pigs.

The M. hyo IDEXX ELISA demonstrated an age effect at day 28. The sixteen weeks pigs seroconverted to the highest level, followed by the sows, and then the 3 week piglets. Both the sows and the 16 week pigs demonstrated a significantly different level of seroconversion when compared with the three week piglet S/P ratios.

### Results

### Effect of Virulence on Immune Responses

The primary parameter of this study is the virus isolation and quantification of the TCID₅₀/mL assay (see FIG. 1). The animals receiving a treatment that contained a virus received equal viral loads. However, the JA142-challenged animals achieved high titers by day 1 of the study. This result confirms the ability of the virulent PRRS isolate to infect and begin in vivo replication rapidly. The titer of the ATP challenged animals began to increase about 2 days after the JA142-challenged animals. In addition, the ATP virus did not prove to be as efficient in the JA142-challenged animals at infection and in vivo replication to high titers. The peak titers for each age group were about 2 logs higher for the JA142 animals than the ATP animals. These results are in keeping with trends identified through previous PRRS studies.

The qPCR results (see FIG. 8) mimicked many of the trends demonstrated in the TCID₅₀ results. The spike in titer began on the day 1 for both the JA142 animals and the ATP animals. These spikes could be due to the fact that this assay cannot differentiate between live and dead virus. But, there is a noticeable difference in the reported copies/mL between the treatment groups. The JA142 animals achieved nearly double the copies/mL by days 1 and 3 than the ATP animals. This relative increase may be due to the JA142 virus's ability to infect and replicate in vivo. The ATP virus was serially passed from its JA142 parent to adapt its affinity towards the MA104 cell culture over the typical swine host. The qPCR data also showed signs of virus in the sera for the duration of the study. Again, this observation is most likely due to the fact that the assay is not able to differentiate between live and dead virus. The qPCR assay also is more sensitive than the TCID₅₀ assay. This difference would leave the group averages slightly higher for the entire study since no “negative” animals are being averaged in with the rest of the respective group.

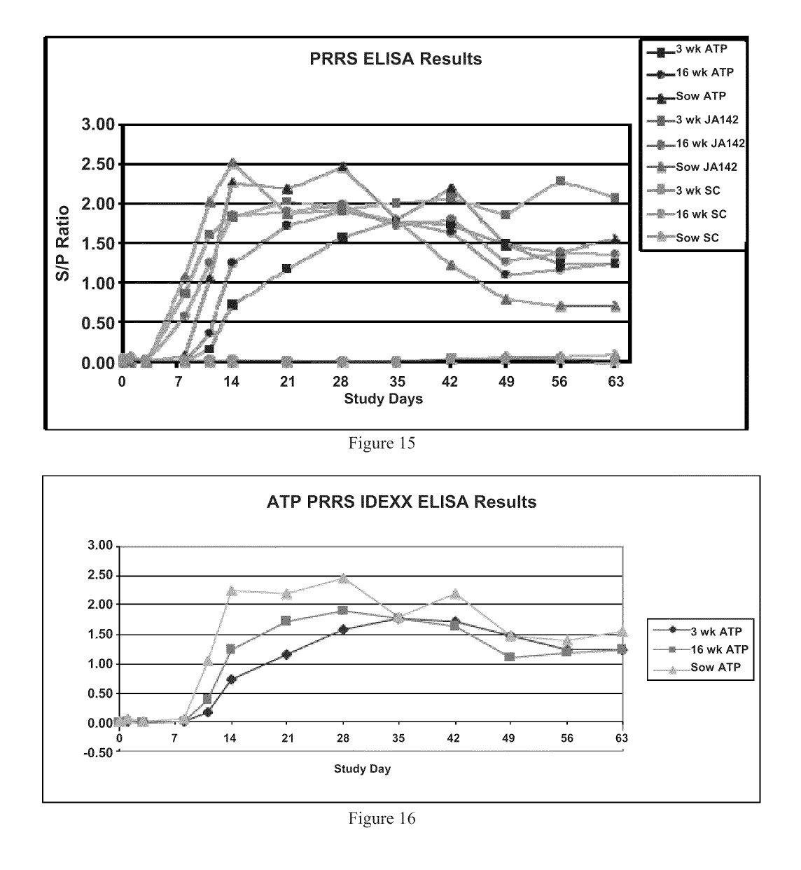

Results from the IDEXX PRRS ELISA (see FIG. 15) revealed a 4 day earlier occurrence for the seroconversion of the JA142 animals over the ATP animals. The JA142 animals began the increase in S/P ratio as of day 3 and peaked around day 14. However, the ATP animals commenced an increase of S/P ratios around day 7 and continued to climb until day 28. These data agree with trends identified in previous PRRS studies. Causes for this response could be that the ATP virus has been serially passed to prefer an artificial, non-swine cell line. The ATP takes longer to infect; therefore, it appears to take longer to induce an immune response. In addition, the JA142 infected isolate replicated to higher numbers faster in vivo than the ATP isolate possibly leading to quicker seroconversion.

### Effect of Animal Age on Immune Responses

When comparing PRRS virus behavior in pigs of varying age, clear differences are illustrated relating to the duration of viremia, overall virus titer, and the speed and level of seroconversion. A slight difference was also noticed within the M. hyo assay.

The TCID₅₀/mL assay shows that the 3 week piglets exhibit the longest duration of viremia (FIG. 1). The 3 week JA142 group gave positive assay results for live virus in the sera beginning on day 1 and lasting through day 42. Likewise, the 3 week ATP piglets proved to have detectable levels of live virus in their sera beginning after day 3 and lasting through the end of the study. In contrast, the assays performed on the sera from the 16 week pigs and the sows demonstrated detectable levels of virus beginning within the first few days and lasting around 2 weeks. The qPCR results (FIG. 8) do not demonstrate differences in the duration of viremia when comparing the different age groups. This result is most likely the result of the qPCR assay being unable to differentiate between live and dead virus. The detection and sensitivity of this assay is unable to demonstrate if any group of animals completely cleared the virus.

The peak TCID₅₀ titers for the 3 week old piglets were 1 to 2 logs higher than the peak titers achieved by the 16 week pigs and the sows. The 3 week JA142 piglets developed an average peak titer of 4.5 logs, whereas the older JA142 animals achieved average peak titers around 2.8 logs. Likewise, the 3 week ATP piglets developed an average peak titer of 3.4 logs while the older ATP animals attained average peak titers around 1.5 logs. The qPCR results (FIG. 8) confirmed these findings. The data demonstrate that the 3 week animals are not as able to clear the virus or control its in vivo replication as efficiently as the older animals. This result may be due to the 3 week piglets having a weaker immune system or the fact that they may have more PRRSV susceptible cells than the older pigs. Both of these viewpoints would increase the virus's ability to infect and replicate to higher levels.

The IDEXX PRRS ELISA results also reveal differences among age groups. The sows seroconverted faster and achieved higher S/P ratios when compared to the 3 week piglets and the 16 week pigs. However, the 3 week piglets and the 16 week pigs show no significance until the end of the study for the JA142-challenged animals only. This difference is most likely due to the immaturity of the 3 week piglet immune system. The young immune system cannot activate its defenses fast enough to initiate fast and adequate seroconversion. This result further validates the idea that the 3 week piglets cannot efficiently control the in vivo replication or clear the virus. The immature immune system of the 3 week old piglets does not protect as well as the fully developed immune system of the 16 week pigs and the sows.

Finally the IDEXX M. hyo ELISA showed slight, non-statistical differences when comparing the S/P ratios among groups. Considering only the data for the 3 bleed days tested, all pigs reached peak S/P ratios on the same day (see FIG. 22). However, a minor difference does exist within the response between the ages. All of the 16 week pigs responded best and are grouped together. They are followed by the sows grouped together, and the 3 week piglets grouped together with the lowest overall S/P ratios. In general, the S/P ratios for the assay were so low that no statistical significance was found.

### Correlations

Correlations derived from comparing group mean results between two different assays rendered expected results. The TCID₅₀ and quantitative PCR results yielded significant, positive correlations between all challenged groups. The correlations further illustrate the consistency within the different assays used to verify the viral behavior. A reason for a less than perfect positive correlation would be that the TCID₅₀ assay can only detect live virus; whereas, the qPCR can detect both live and dead virus in sera. Comparisons between both the TCID₅₀ and qPCR with the PRRS ELISA data yielded non-significant r values near or less than zero. This conclusion is the result of the virus replication and persistence behavior in vivo differing from the seroconversion behavior detected by the ELISA. Finally, the gross lung lesions and the PRRS specific IHC lung scores have an expected significant, positive overall correlation.

The area under the curve (AUC) data correlations demonstrate the “percent probability that a randomly selected observation in one group is greater than a randomly selected observation in the other group.” These data show that there is at least a significant 57% chance that any one of the 3 week ATP piglets will have a higher titer than any other pig in the study. There is also at least a statistically significant chance that any one of the JA142-challenged animals will have a higher titer than any other pig in the study. Finally, the strict controls yield a non-significant percentage of 50 when compared with other strict control pigs. These values remained zero for the duration of the study.

### Statistical Correlations

Comparisons of the group means for the TCID₅₀, qPCR, and PRRS ELISA assays were made. Spearman coefficients of 1 show a positive correlation whereas a correlation coefficient of −1 shows a negative correlation. A significant p value <0.05 demonstrates a significant correlation, but not a significant difference. Table 1 illustrates the significant, positive correlation between the TCID₅₀ and the qPCR results among all experimental groups. The TCID₅₀ and the PRRS ELISA appear to have negative correlation. But, this correlation is not significant. Finally, the gross lung lesions and the PRRS specific microscopic lung lesions have a significant, positive correlation.

TABLE 1
Group Mean Assay Correlations

| | | | TCID₅₀ and PRRS | | qPCR and PRRS | | Gross vs Micro. | |
|---|---|---|---|---|---|---|---|---|
| | TCID₅₀ and qPCR | | ELISA | | ELISA | | Lungs | |
| | | P Value | | P Value | | P Value | | P Value |
| Detected | Scc#¹ | for Sc | Scc# | for Sc | Scc# | for Sc | Scc# | for Sc |
| Overall | 0.7303 | <0.0001* | 0.5175 | <0.0001* | 0.5175 | <0.0001* | 0.9273 | 0.0003* |
| Group 1 | 0.8413 | 0.0003* | 0.5635 | 0.0449* | 0.5635 | 0.0449* | — | — |
| Group 2 | 0.6183 | 0.0243* | −0.1116 | 0.7167 | −0.1116 | 0.7167 | — | — |
| Group 3 | 0.6612 | 0.0139* | 0.0707 | 0.8184 | 0.0707 | 0.8184 | — | — |
| Group 4 | 0.812 | 0.0007* | −0.4077 | 0.1667 | −0.4077 | 0.1667 | — | — |
| Group 5 | 0.7181 | 0.0057* | −0.1667 | 0.5863 | −0.1667 | 0.5863 | — | — |
| Group 6 | 0.5871 | 0.0349* | 0.2493 | 0.4114 | 0.2493 | 0.4114 | — | — |
| Group 7 | — | — | — | — | — | — | — | — |
| Group 8 | — | — | — | — | — | — | — | — |
| Group 9 | — | — | — | — | — | — | — | — |

¹Scc# = Spearman correlation coeficient number
²Sc = Spearman coeficient
³#= Value of 1 is a perfect positive correlation, value of −1 is a perfect negative correlation
⁴*= Significant at ≦0.05 level
⁵— = No Comparison

Receiver Operator Curve (ROC) analysis was performed on the TCID₅₀ data to determine the area under the curve (AUC) and results are shown in Table 2, which reads group X axis verses group Y axis. The AUC is the “percent probability that a randomly selected observation in one group is greater than a randomly selected observation in the other group.” The notation of a p value <0.05 denotes that the percent probability is significant.

TABLE 2
Group Mean TCID50 Area Under the Curve (AUC) Correlations

| Group | 1 | 2 | 3 | 4 | 5 | 6 | 7 | 8 | 9 |
|---|---|---|---|---|---|---|---|---|---|
| 1 | — | 0.67* | 0.65* | 0.652* | 0.608* | 0.574* | 0.696* | 0.696* | 0.696* |
| 2 | 0.67* | — | 0.523 | 0.767* | 0.566 | 0.601* | 0.531 | 0.531 | 0.531 |
| 3 | 0.65* | 0.523 | — | 0.754* | 0.543 | 0.579* | 0.554 | 0.554 | 0.554 |
| 4 | 0.652* | 0.767* | 0.754* | — | 0.729* | 0.709* | 0.783* | 0.783* | 0.783* |
| 5 | 0.608* | 0.566 | 0.543 | 0.729* | — | 0.535 | 0.596* | 0.596* | 0.596* |
| 6 | 0.574* | 0.601* | 0.579* | 0.709* | 0.535 | — | 0.628* | 0.628* | 0.628* |
| 7 | 0.696* | 0.531 | 0.554 | 0.783* | 0.596* | 0.628* | — | 0.5 | 0.5 |
| 8 | 0.696* | 0.531 | 0.554 | 0.783* | 0.596* | 0.628* | 0.5 | — | 0.5 |
| 9 | 0.696* | 0.531 | 0.554 | 0.783* | 0.596* | 0.628* | 0.5 | 0.5 | — |

*denotes P Value significant at p ≦ 0.05

Table 2 indicates that the JA142 animals (groups 4-6) are likely to have a significantly higher TCID₅₀ titer than a large majority of the groups. Also, the 3 week animals (groups 1, 4, and 7) are more likely to have significantly higher titers that the majority of the other groups in the study. Finally, there is a 50% chance that one of the control animals will have a higher titer than another control animal. These values are non-significant; however, these pigs' assay results had identical values for the duration of the study.

In one embodiment, the consequences of PRRSV infection are highly dependent on pig age. Viral growth is most extensive in piglets. For both virulent and attenuated PRRSV, peak viremia and duration are substantially greater in piglets. Finishers and sows show the same pattern of low level viremia for virulent viral infection that resolved within 2 weeks and approximately 50% of finishers and sows inoculated with attenuated PRRSV showed no viremia. The prolonged period of viremia commonly associated with PRRSV infection is based on studies in young pigs. Unexpectedly, viremia was found to be substantially reduced in growing and adult pigs indicating in one embodiment that the mechanisms of PRRSV resistance are developmentally regulated. Attenuated or lowly virulent PRRSV grow poorly in young pigs, but the frequent absence of viremia in older age pigs has not been documented previously.

In another aspect, the restriction in viral growth in older pigs is due to differences in innate immunity or in host cell permissiveness. Since as disclosed in example 5, onset of viremia is the same or earlier in finishers and sows compared to piglets, permissive macrophages are available at all times. Acute infection of pigs at about 20 weeks of age does not reduce the abundance of macrophages in lung or lymphoid tissues. Therefore, in another embodiment, suppression of PRRSV infection in older animals is due to more potent mechanisms of innate resistance. In another embodiment, PRRSV selectively induces an immunosuppressive response that blocks innate resistance in young pigs.

mRNA levels or secreted cytokines implicate IL-10 induction by PRRSV infection as a mechanism facilitating viral persistence. As described herein, IL-10 concentrations are significantly and transiently elevated as much as up to 40 pg/mL in serum of piglets infected with virulent PRRSV. However, older pigs frequently exhibit higher levels of IL-10, up to 800 pg/mL, before infection and when uninfected. Since the level of IL-10 prior to infection had no effect on the level of viremia, since peak viremia occurred in piglets one week before the appearance of IL-10, and since it was not observed in piglets exposed to attenuated PRRSV, in one embodiment, IL-10 production in piglets is a direct and predictive consequence of viral virulence and pathogenesis, rather than being the cause of viral persistence.

Accordingly and in one embodiment provided herein is a method of detecting a virulent PRRSV infection in piglets comprising the step of monitoring the levels of IL-10 in blood serum of said piglet, wherein an increase in IL-10 concentration up to 40 pg/mL indicates a virulent PRRSV infection. In another embodiment provided herein is a method of differentiating between viral persistence and viral pathogenesis in pigs comprising the steps of determining the age of the pig, obtaining a blood serum sample from the pig and determining the serum concentration of IL-10 in the blood serum, wherein if the pig is a less than 8 weeks old the presence of IL-10 concentration up to 40 pg/mL indicates virulent pathogenesis and not viremia persistence. In one embodiment provided herein is a method of gauging the effect of anti-viral treatment in piglets or screening anti-viral compositions comprising the steps of administering a candidate composition to the piglet and monitoring the level of IL-10 in the blood serum of the piglet, wherein the composition capable reducing IL-10 levels to the lowest level in the shortest time is the most effective.

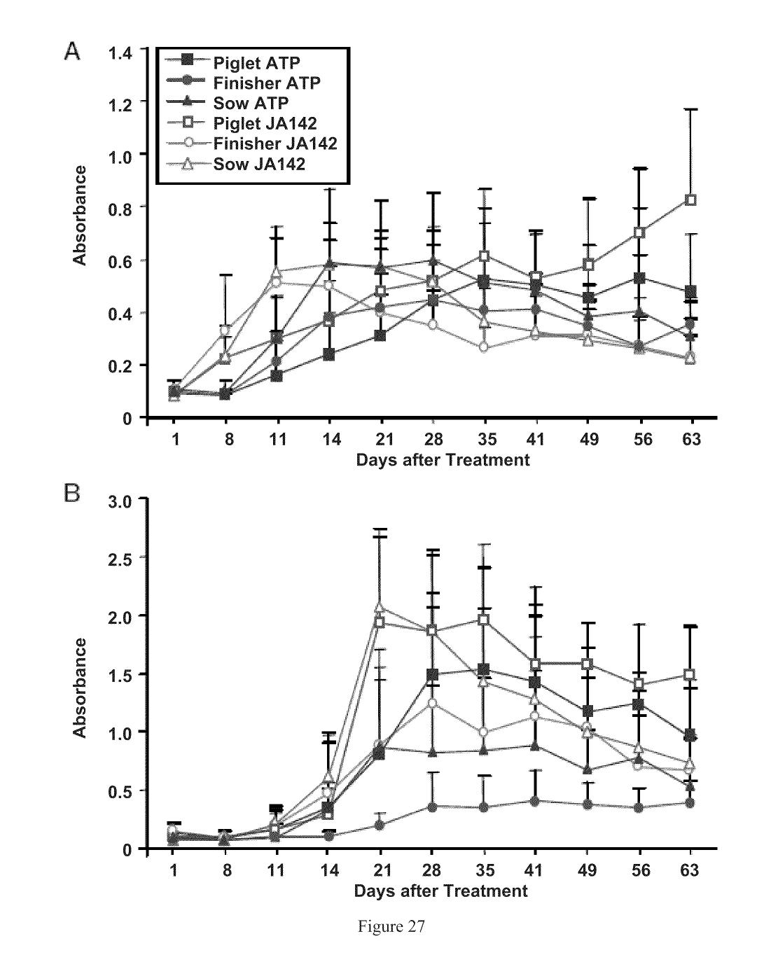

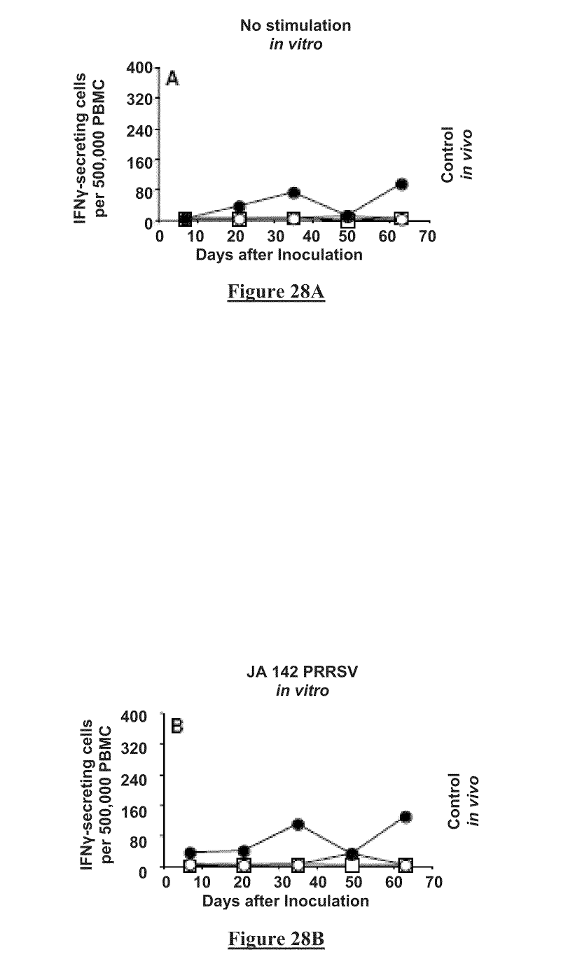

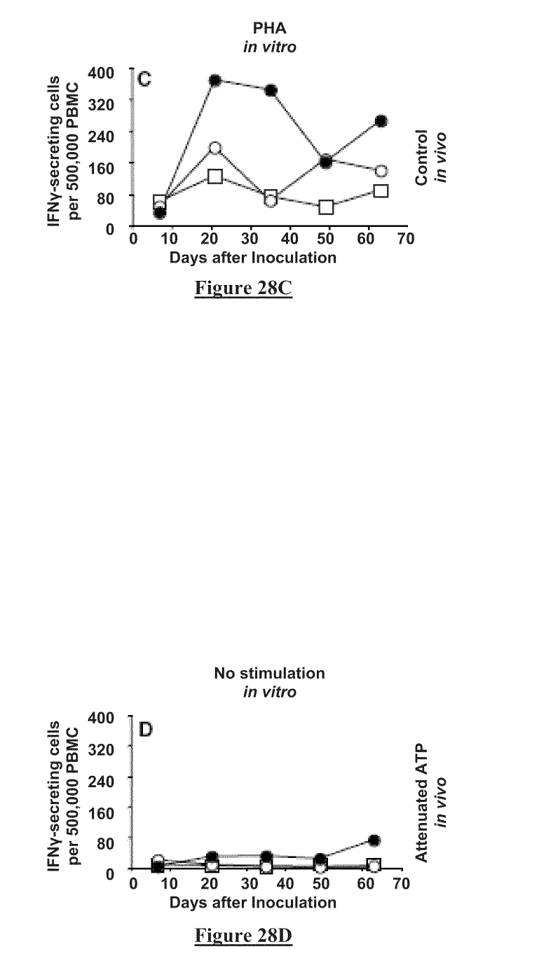

In contrast to their effects on infection, pig age and viral virulence had relatively little impact on the antigen-specific adaptive immune response, even though viremia was not observed in nearly half (9/20) of juvenile and adult pigs. Regardless of the viral strain used to challenge pigs, all animals seroconverted, and all groups showed the same level of antibody by HerdChek® PRRS 2XR ELISA at day 35. Variation in the intensity of antibody responses appear to be random since differences in kinetics or intensity of response determined by one assay are not reproduced when the same sera is analyzed by another assay as demonstrated by comparison of the group responses to N and nsp2Hp in FIG. 27.

In one aspect, antigen-specific immunological competence is achieved in pigs by day 74 of gestation, i.e. midway in fetal development. At 3 weeks of age, piglets show strong IgM and IgG antibody responses to the protein antigen, keyhole limpet hemocyanin, and a variety of PRRSV proteins following infection. Therefore, even if antigen-specific adaptive immunity is not fully developed in piglets, the failure to achieve more rapid elimination of viremia does not appear to be related to the adaptive immune response.

Molecular and cellular mechanisms of innate immunity to viral infection are extensive, but little is known about their role in resistance to PRRSV infection. Cellular immunity mediated by NK cells or other cell types has not been explored. Absence of IFNα induction early in infection is well described and believed to help explain prolonged infection. No comparative studies of differences in interferon responses or other innate immune mechanisms exist that might explain the marked age-dependent differences in infection outcomes in young versus older pigs. Interleukin-10, which has been suggested to suppress anti-PRRSV immunity, has been shown to suppress inflammatory cytokine production and reduce disease severity in a swine model of bacterial pleuropneumonia. In another embodiment, IL-10 production is pathognomonic of virulent infection rather than a cause of prolonged infection.

In another embodiment, differences in circulating IFNγ secreting cells do not account for differences in age-dependent infection. Rather, they indicate that finishers are more similar to piglets, a conclusion that is in contrast to the similarity between finishers and sows in control of viral infection. The interpretation of IFNγ secreting cell frequencies is confounded since IFNγ in pigs is produced by a wide variety of cell types, including activated CD8+T cells, natural killer T cells, and γδT cells, in addition to type 1 CD4+T cells.

The lack of a substantial effect of pig age on antigen-specific immune responses in contrast to a significant age dependent effect on the kinetics of infection supports the concept that control of PRRSV viremia may not be dependent on adaptive immune responses. In one embodiment, infection is controlled though not eliminated by a deficiency in permissive macrophages. A similar phenomenon operates in another embodiment to control PRRSV viremia, which occurs before neutralizing antibody responses are observed. This occurs in one embodiment through interference with virus binding to its CD163 receptor on macrophages.

In one embodiment, all groups of pigs exposed to PRRSV develop equivalent adaptive antibody and cell mediated immune responses irrespective of the kinetics or magnitude of viremia. This indicates that the requirements of antigenic mass and mode of presentation for an immune response to PRRSV are met at a low level of infection in the absence of viremia. In another embodiment, viremia is an insensitive indicator of infection by lowly virulent or attenuated PRRSV strains, especially in growing and mature swine. In one embodiment, resolution of viremia does not require an adaptive immune response. While adaptive immunity most likely is essential for protection against future challenge, control of primary infection relies in one embodiment on innate mechanisms of immunity that are more effective at about 15 weeks of age and older.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should appreciate that other equivalent techniques known in the art may be used and still obtain a like or similar result.

### EXAMPLES

On Day 0, a PRRS vaccine strain, a PRRS challenge strain, or a media only placebo (determined by group) was administered to each pig.

The study consisted of six challenge groups and three control groups. All groups (Groups 1-9) had ten animals. Groups seven through nine served as challenge controls and only received placebo on day 0. All animals were inoculated with the respective PRRSV isolate, or placebo on day 0. All animals in all groups were also vaccinated with a killed M. hyo vaccine on day 0. The nine groups are listed in Table 3. Blood samples were collected on days 0, 1, 3, 8, 11, 14, 21, 28, 35, 42, 49, 56, and 63. On day 63, the study was terminated, and all animals were humanely euthanized and necropsied.

TABLE 3
Treatment Schedule.

| | N | First Challenge (Day = 0) | Sample collection and study |
|---|---|---|---|
| Group | (Age) | 2 mL administered nasally | termination (Day = 63) |
| 1 | 10 | Ingelvac ® PRRS ATP | Evaluate clinical health, rectal |
| | (3 weeks old) | | temperature, and collect blood |
| | | | samples. Evaluate animals for lung |
| | | | lesions at necropsy and collect |
| | | | designated lung tissue. |
| 2 | 10 | Ingelvac ® PRRS ATP | Same as above |
| | (16-20 weeks old) | | |
| 3 | 10 | Ingelvac ® PRRS ATP | Same as above |
| | (3^{rd} ± 1 parity sows) | | |
| 4 | 10 | JA142 | Same as above |
| | (3 weeks old) | | |
| 5 | 10 | JA142 | Same as above |
| | (16-20 weeks old) | | |
| 6 | 10 | JA142 | Same as above |
| | (3^{rd} ± 1 parity sows) | | |
| 7 | 10 | MEM with 4% FCS | Same as above |
| | (3 weeks old) | | |
| 8 | 10 | MEM with 4% FCS | Same as above |
| | (16-20 weeks old) | | |
| 9 | 10 | MEM with 4% FCS | Same as above |
| | (3^{rd} ± 1 parity sows) | | |

### Materials:

Materials were prepared and administered as described below in Table 4.

TABLE 4

| Challenge Isolate | Virulent or Vaccine Isolate of PRRSV (as per group) |
|---|---|
| Challenge preparation | On day 0, the PRRS Isolate was diluted in Modified Eagles |
| | Medium containing 4% Fetal Calf Serum. Diluted challenge |
| | virus was equal to log 3.0 +/− 0.5 per ml. |
| Dose of Challenge material | 1 × 1 mL dose intranasal (1 ml in one nostril) and 1 × 1 ml |
| | dose intramuscular in the shoulder/neck region. |
| Handling of Challenge Material | Challenge material was kept on ice prior to administration to |
| | the test animals and during the challenge procedure. |
| Testing of Challenge Material | Diluted challenge virus was titrated on 96-well plates |
| | containing three-day-old CL2621 cells. |
| Method of Administration | intranasal and intramuscular. |
| Schedule of Challenge Treatment | Day 0. |
| Frequency of Administration of | Once. |
| Challenge Material | |

### TCID₅₀ Assay/Virus Isolations

Serum was separated from clotted whole blood by centrifugation at 6000 RPM for 20 minutes. One hundred microliters (μL) of serum was added to a dilution tube containing 900 μL of Eagle's Minimum Essential Media (EMEM)+2% Fetal Bovine Serum (FBS)+100 units/mL of Penicillin+100 μg/mL Streptomycin+2.5 μg/mL Fungizone. This tube was vortexed and 100 μL was transferred to another dilution tube containing 900 μL of EMEM+2% FBS+100 units/mL of Penicillin+100 μg/mL Streptomycin+2.5 μg/mL Fungizone. The process was repeated until a dilution of 10-6 was reached. Each dilution was plated on 96-well plate containing MA 104 cells, 100 μL per well, four replicates for each dilution. The plates were incubated at 37° C. with 4% CO₂ for eight days and then each well was examined for CPE. The titer was determined using the Reed-Muench calculation (VBFO).

One hundred μL of serum was added to each of duplicate test wells containing MA 104 cells. The plates were then incubated for one hour at 37° C. with CO₂. Next 500 μL of EMEM+2% FBS+100 units/mL of Penicillin+100 ug/mL Streptomycin+2.5 ug/mL Fungizone was added to each well. The plates were incubated at 37° C. with 4% CO₂ for eight days and then each well was examined for CPE.

### Quantitative PCR

RNA Extraction from Serum.

To obtain viral RNA, the QIAamp Viral RNA Mini-Kit® was used as described in the kit instructions.

Real-Time PCR.

A commercially available real-time, single-tube, RT-PCR assay for the detection of U.S. or LV/European-like PRRSV was provided by Tetracore Inc. (Gaithersburg, Md.) and used to detect PRRSV RNA. A minor groove binding (MGB) 5′ nuclease probe and primers were designed from the 3′ UTR PRRSV genomic region by alignment of GenBank isolates and based on conserved areas of the 3′ UTR primer and probe region. The PRRSV RNA was transcribed in a single tube using a 25 μL reaction volume consisting of Tetracore U.S. PRRSV Master Mix [18.9 μL Master mix, 2 μL Enzyme mix 1, 0.1 μL Enzyme mix 2] (Tetracore, Inc., Rockville Md.) and 4 μL of extracted RNA. The reaction tubes were loaded into the Smart Cycler II® block (Cepheid, Sunnyvale, Calif.), and software settings of fluorescent detection were set for automatic calculation of the baseline with the background subtraction on. The thermal cycler program for the U.S. PRRSV real-time RT-PCR assay consisted of 52° C. for 1800 s, 95° C. for 900 s, and 45 cycles at 94° C. for 30 s, 61° C. for 60 s and 72° C. for 60 s. For the LV/European-like PRRSV assay, thermal cycling times consisted of 60° C. for 1200 s, 95° C. for 15 s and 45 cycles at 95° C. for 3 s, 60° C. for 30 s. Copy numbers are reported for the full 45 cycles. A PCR reaction was considered positive if the cycle threshold (Ct) level was obtained at ≦36 cycles and suspect if a Ct was obtained between 37 and 39 cycles.

### Serology (IDEXX PRRS ELISA and IDEXX M. hyo ELISA)

Serological studies were performed as described in kit instructions. Blood was collected from animals for PRRS virus serology and virus quantification on Day 0, 1, 3, 8, 11, 14, 21, 28, 35, 42, 49, 56, and 63.

### Body Weights

All pigs were weighed on Day 0 (first day of study) and Day 63 (day of necropsy). The 3 week pigs and the 16 week pigs were also weighed on day 28 of the study. Pigs were weighed on an electronic weighbar scale system (Weigh-Tronix™, Weigh-Tronix Inc., Fairmont, Minn.) that was calibrated using certified test weights prior to and after each use.

### Clinical Scores

On every day of the study each pig's clinical health was scored on the following three criteria: respiratory, behavior, and cough. The score from each criterion can range from one to four, with a normal animal given a score of three, maximum clinical illness given a score nine and a dead animal given a score of 12. Any abnormal clinical findings also were recorded.

### Gross Lung Lesions

At the time of necropsy, the lungs of each pig were removed, examined and scored for the presence of gross lesions. The individual lobes were examined, scored separately, and the scores were then combined to give a total percent of lung lesions present (PigMon scoring).

### Immunohistological Evaluation of Lungs

A sample of lung from each pig was fixed in 10% formalin on the day of necropsy and tested by immunohistochemistry and microscopic examination for staining and lesions compatible with PRRSV, respectively. This testing was performed by the Iowa State University Veterinary Diagnostic Lab.

### Day of Necropsy

Pigs were bled at study end. Many pigs were necropsied on day 63. Necropsies were finished on day 64. During necropsy 0.5 g samples of the spleen, inguinal lymph node, bronchial lymph node, and tonsil, respectively, were collected for later RNA analyses.

The purpose of this study was to evaluate the impact of age and immune response on PRRS in vivo replication, persistence, and ability to cause disease. Viral isolates with known different virulence levels were used to innoculate animals of different ages. Animals were monitored to assess their B cells and immune responses. Immune responses were elicited and evaluated among three different age groups, each of which received three different treatments of varying virulence. The TCID₅₀/mL assay, the quantitative RT-PCR, and the PRRS IDEXX ELISA data generated the most significant conclusions. Due to the duration of the study without a challenge, the remaining secondary parameters yielded trivial data sets for application to future vaccine development and trials. Results are described in the following examples.

### EXAMPLE 1

### Log TCID₅₀/mL Assays

Two PRRS isolates with known different virulence levels were used to inoculate animals of varying age, and the immune responses of these animals were evaluated for differences. The viremia data was used to correlate B cells and immune responses. Finally, to check for immune interference, all pigs were vaccinated with a killed M. hyo vaccine as well.

The TCID50 data shows several apparent trends with the effects of age and virulence on the level of viremia in pigs (summarized in FIG. 1). There is a distinct delay in viremia in pigs of all ages receiving the Ingelvac® PRRS ATP when compared to those pigs receiving the virulent JA142 challenge strain. Also, the young pigs achieved higher levels of viremia and retained those higher levels longer than both of the older groups of pigs.

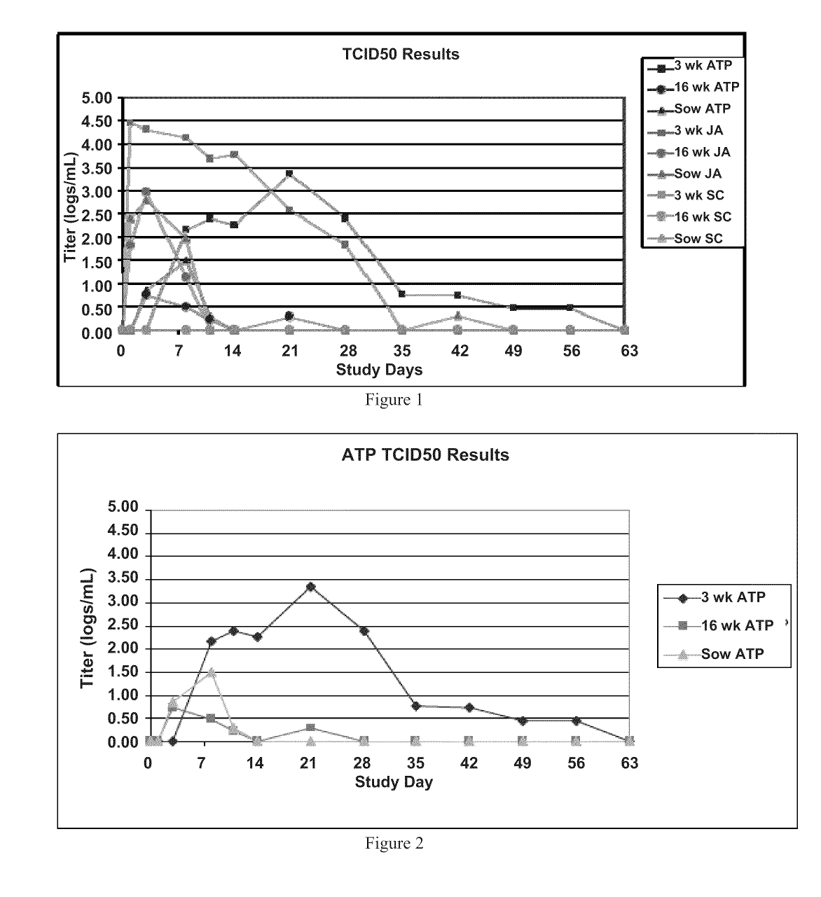

FIGS. 2-7 better illustrate these observations. Statistics were performed by completing a group average non-parametric ANOVA. The data achieving significant values (Kruskal-Wallis p<0.05) were re-evaluated pairwise using the Wilcoxon Two-Sample Test (p<0.05.)

### Challenge Material Titers

Table 5 below shows for the pre and post exposure titers of the challenge material. Desired inoculation was 3.0±0.5 Log TCID₅₀.

TABLE 5
Pre/Post Exposure and Challenge Titer Raw Data

| | | | | | | Ave Log |
|---|---|---|---|---|---|---|
| TEST ARTICLE: JA 142 | | | | | | TCID₅₀ |
| Before Inoculation | 3.47 | 3.25 | 2.88 | 3.36 | 2.58 | 3.11 |
| After Inoculation | 3.16 | 3.25 | 2.88 | 2.60 | 3.40 | 3.06 |
| | rep 1 | rep 2 | rep 3 | rep 4 | rep 5 | |
| | | | | | | Ave Log |
| TEST ARTICLE: Ingelvac ® ATP (ATP) | | | | | | TCID₅₀ |
| Before Inoculation | 2.50 | 2.84 | 2.75 | 2.64 | 3.25 | 2.80 |
| After Inoculation | 2.75 | 2.75 | 2.88 | 3.25 | 3.25 | 2.98 |
| | rep 1 | rep 2 | rep 3 | rep 4 | rep 5 | |

The Ingelvac® ATP 16 week pig and sow groups show an increase in titer starting on day 3 whereas the 3 week piglets do not show a titer until day 8 (see FIG. 2). The two older groups achieve peak titers within the first week of inoculation and clear the virus at the end of the second week. The 3 week piglets reach their peak titer at day 21 and maintain a titer through the end of the study. The Wilcoxon Two-Sample Test (p<0.05) data from days sampled show that the 3 week ATP titers are significantly different from the 16 week ATP titers on days 8 through 28 and significantly different from the sow ATP titers on days 11 through 28. The 16 week ATP and the sow ATP titers are not significantly different from one another on any day of the study.

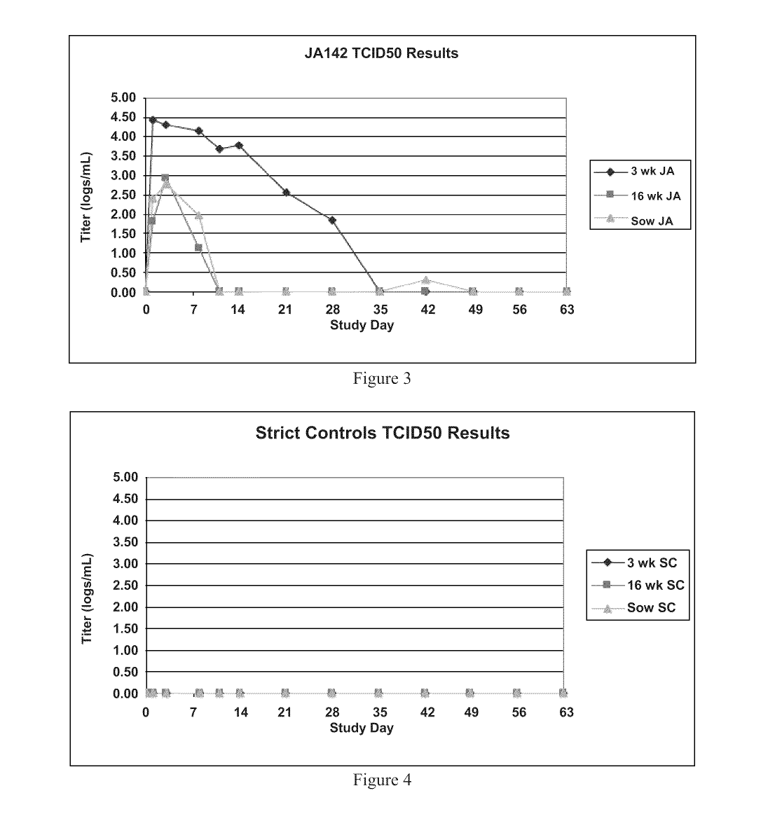

All JA142 groups show increases in titers on day 1 of the study (see FIG. 3). The 16 week JA142 pigs and the JA142 sows obtain peak titers on day 3 and appear to clear the virus by day 11. The titers for the 3 week JA142 piglets peak on day 1 and remain detectable until day 35. The Wilcoxon Two-Sample Test (p<0.05) data from days sampled show that the 3 week JA142 titers are significantly different (i.e. higher) from the 16 week JA142 and JA142 sow titers on days 1 through 28. The 16 week ATP and the sow ATP titers are only significantly different from one another on day 1 of the study.

All controls remained negative for live virus for the duration of the study (see FIG. 4). The statistical analysis revealed no days with significant differences.

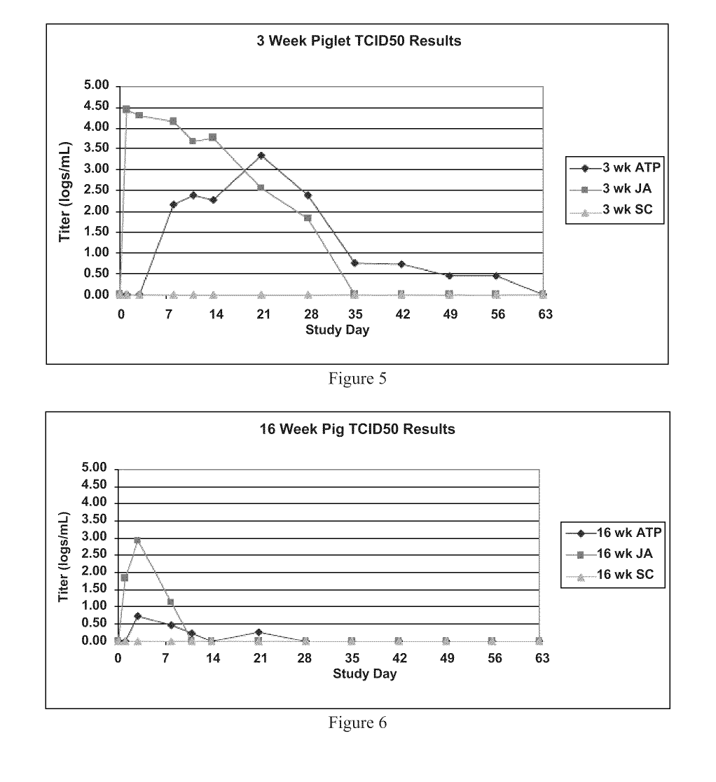

The 3 week ATP piglets showed an increase in titers beginning on day 3, obtained a peak titer on day 21, and maintained a titer throughout the study (see FIG. 5). The 3 week JA142 piglets obtained peak titers on day 1 and cleared the virus by day 35 of the study. The 3 week control piglets remained below a detectable level of virus for the duration of the study. The Wilcoxon Two-Sample Test (p<0.05) data from days sampled show that the 3 week ATP titers are significantly different from the 3 week JA142 titers on days 1 through 14. The 3 week ATP titers are also significantly different from the 3 week control pigs on days 8 through 28. Finally, the 3 week JA142 titers and the 3 week control titers are significantly different on days 1 through 28.

The 16 week ATP titers began to increase on day 3 and were no longer detectable as of day 28 (see FIG. 6). The 16 week JA142 titers increased immediately, peaked on day 3, and cleared from the serum as of day 11. The 16 week control titers remained at an undetectable level for the duration of the study. The Wilcoxon Two-Sample Test (p<0.05) data from days sampled show that the 16 week JA142 titers are significantly different from both the 16 week ATP titers and the 16 week control titers on days 1 and 3. The 16 week ATP titers and the 16 week control titers showed no days of statistical significance from one another.

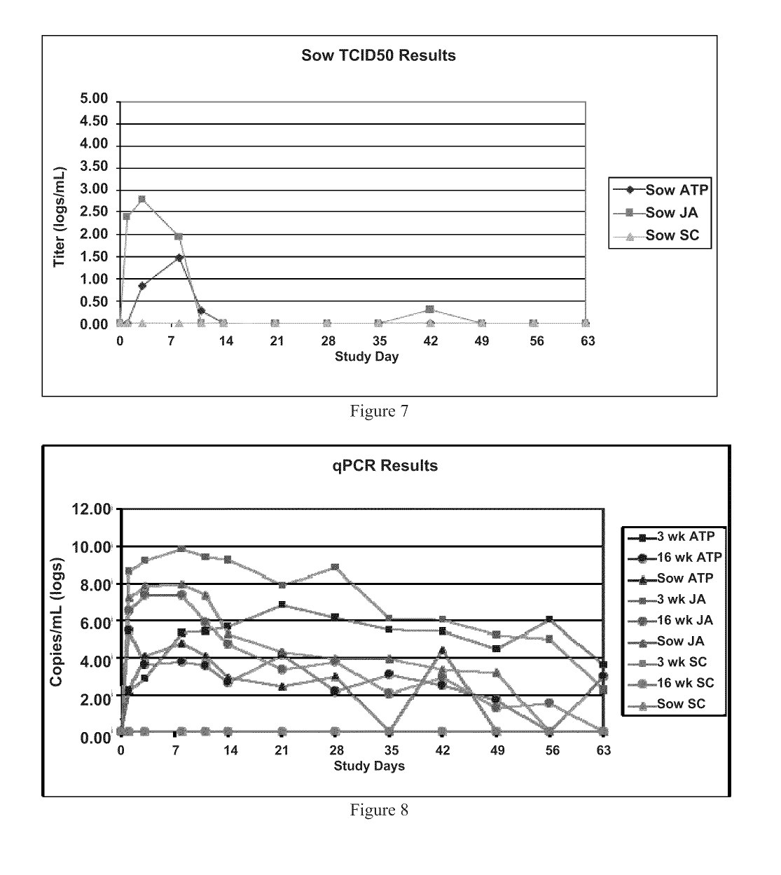

The ATP sow titers showed an increase as of day 3 and were no longer at a detectable level as of day 14 (see FIG. 7). The JA142 sow titers increased immediately, peaked at day 3, and remained low or undetectable as of day 11. The control sow titers remained undetectable for the remainder of the study. The Wilcoxon Two-Sample Test (p<0.05) data from days sampled show that the ATP sow titers are significantly different from the JA142 sow titers on days 1 and 3. The ATP sow titers are also significantly different from the control sow titers on days 3 and 8. Finally, the JA142 sow titers are significantly different from those of the control sows on days 1 through 8.

### EXAMPLE 2

### Quantitative PCR (Copies/mL) Assays

The quantitative PCR (qPCR) data also shows a couple trends in relation to age and virulence (summarized in FIG. 8). The JA142 group achieves a higher copy number of virions per milliliter (mL) of serum when compared to the Ingelvac® ATP and control groups. Also, the 3 week piglets in each treatment have higher virus copies per mL than the sows. Likewise, the sows have a greater viral load than the 16 week pigs.

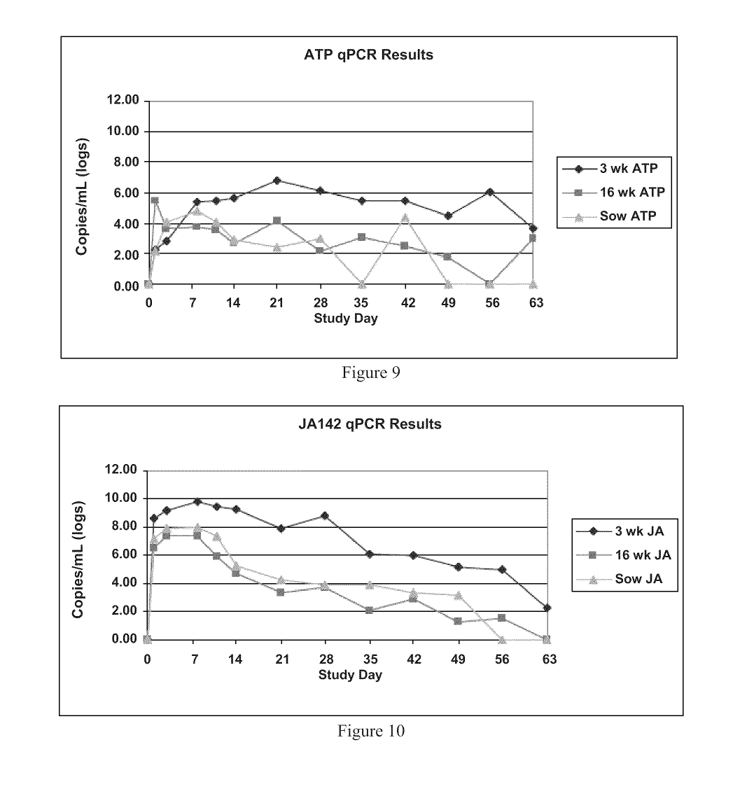

These observations are illustrated in FIGS. 9-15. Statistics were performed by completing a group average non-parametric ANOVA. The data achieving significant values (Kruskal-Wallis p<0.05) were re-evaluated pairwise using the Wilcoxon Two-Sample Test (p<0.05.)

All age groups of ATP treated pigs increase in copy numbers at about the same time (see FIG. 9). The 3 week ATP piglets climb to the highest viral load and maintain 6 logs of viral copies/mL. The two older groups reach a level of about 5 logs and steadily decrease over time. The Wilcoxon Two-Sample Test (p<0.05) data from days sampled show that 3 week ATP piglets have significantly higher copy numbers than the 16 week ATP pigs on days 11 through 42 and days 56 and 63. The 3 week ATP piglets also have significantly different copy numbers than the ATP sows on days 3 and 14 through 63. Finally, the 16 week ATP pigs and the ATP sows are significantly different on day 3 only.

All the pigs receiving the JA142 treatment follow the same trend, varying only in actual copy number (see FIG. 10). The 3 week JA142 piglets have about 2 logs more copies of the virus than the JA142 sow. The JA142 sows have about 1 log more virus than the 16 week JA142 pigs. The Wilcoxon Two-Sample Test (p<0.05) data from days sampled show that the 3 week JA142 piglets have significantly higher copies/mL of virus on days 1 through 49 when compared to either of the two older groups of JA142 animals. When comparing the 16 week JA142 pigs to the JA142 sows, the copy numbers are only significantly different on day 1.

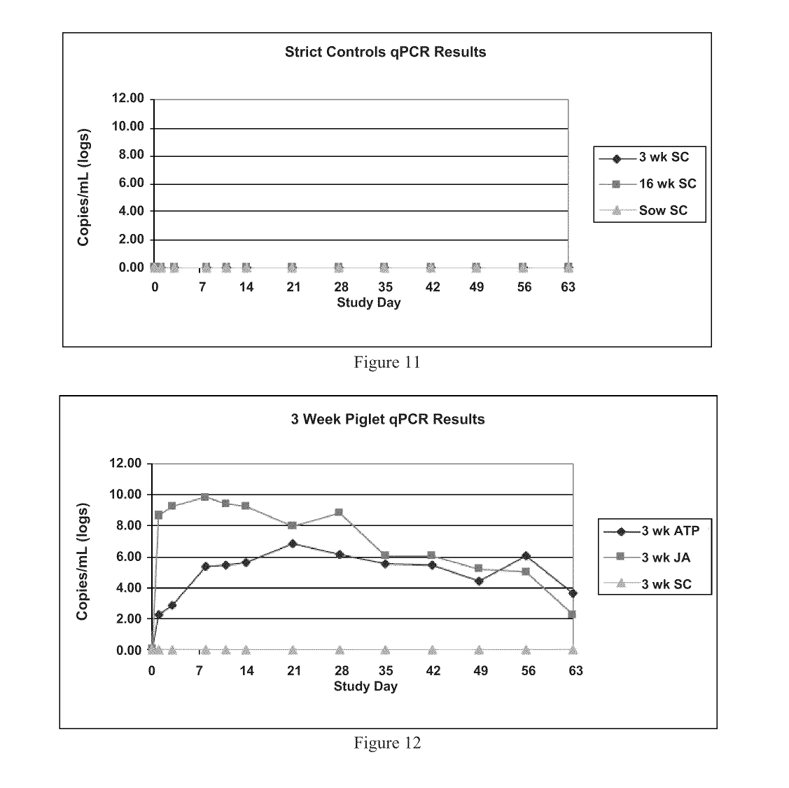

All controls remained negative for virus in the serum for the duration of the study (see FIG. 11). The statistical analysis revealed no days of significant differences.

Both the 3 week ATP and 3 week JA142 piglets spiked in virus copy number as of day 1 (see FIG. 12). However, the JA142 group achieved about 6 logs copies/mL of virus more than the ATP group on the same day. The 3 week ATP piglets steadily climbed to about 7 log copies/mL of virus until day 21, then slowly decreased to 4 logs by the end of the study. The 3 week JA142 piglets peaked at 10 log copies/mL of virus on day 8, then decreased to 2 log copies/mL of virus on day 63. The controls maintained an undetectable level of virus in serum for the duration of the study. The Wilcoxon Two-Sample Test (p<0.05) data from days sampled show that the 3 week ATP piglets had significantly lower log copies/mL than the 3 week JA142 piglets on days 1 through 28. The 3 week ATP piglets also had significantly higher log copies/mL of virus when compared to the 3 week strict controls on days 3 through 63. Finally, the 3 week JA142 piglets had significantly higher log copies/mL of virus than the 3 week control piglets on days 1 through 56.

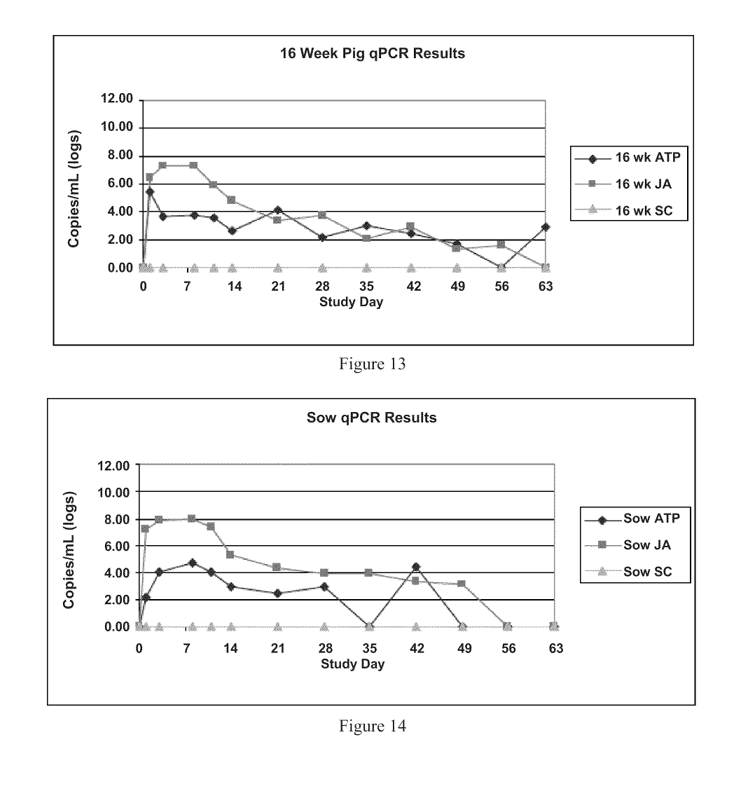

The 16 week ATP pig group spikes on day 1 with the 16 week JA142 pig group, but the 16 week JA142 values continue to climb for the following 2 bleeds and the 16 week ATP pigs achieved decreasing numbers (see FIG. 13). The 16 week controls maintained an undetectable level of virus in serum for the duration of the study. The Wilcoxon Two-Sample Test (p<0.05) data from days sampled show that the 16 week ATP pigs and 16 week JA142 pigs have significantly different viral copies/mL of sera on days 1 through 18. The 16 week ATP pigs and the 16 week control pigs have significantly different viral copies/mL for days 3 through 14. Finally, the 16 week JA142 pigs and the 16 week control pigs have significantly different numbers on days 1 through 28.

Both of the challenged groups have viremic spikes on day 1 and peak copies/mL around day 7. However, the JA142 sows attain a higher number of copies/mL than all of the other groups (see FIG. 14). Important to note is that the sow ATP spike on day 42 was the results of only one pig. As seen with all the control groups, the control sows maintained an undetectable level of virus in serum for the duration of the study on days tested. The Wilcoxon Two-Sample Test (p<0.05) data from days sampled show that the ATP sows and the JA142 sows have significantly different results on days 1 through 21, 35, and 49. The ATP sows are only significantly different from the strict control sows on day 3 through 11. Finally, the JA142 sows are significantly different than the control sows on every day sampled, 1 through 49.

### EXAMPLE 3

### Serology—IDEXX PRRS ELISA (S/P Ratios) Assays

The IDEXX PRRS ELISA data (see FIG. 15) illustrates a couple trends in relation to age and virulence. The JA142-challenged group started to seroconvert about 4 days prior to the Ingelvac® ATP challenged group. Also, the sows in each treatment generally seroconverted quicker and with higher S/P ratios than the 16 week pigs, which generally seroconverted quicker and with higher S/P ratios than the 3 week piglets in their respective challenge groups.

FIGS. 16-21 illustrate these observations. Statistics were performed by completing a group average non-parametric ANOVA. The data achieving significant values (Kruskal-Wallis p<0.05) were re-evaluated pairwise using the Wilcoxon Two-Sample Test (p<0.05.)

When comparing all of the animals challenged with ATP by age group, the general trend for seroconversion was consistent. S/P ratios began to increase on the same day, peaked on the same day, and followed similar patterns after peak ratios were reached. Overall, the sows had higher S/P ratios than the 16 week pigs, and the 16 week pigs had higher ratios than the 3 week piglets (see FIG. 16). The Wilcoxon Two-Sample Test (p<0.05) data from days sampled show that 3 week ATP ratios are not significantly different from the 16 week ATP ratios on any of the sample days. However, the 3 week ATP piglets have significantly different S/P ratios than the ATP sows on days 8 through 28. Similarly, the 16 week ATP pigs have significantly different values than the ATP sows on days 8 through 14 and 28.

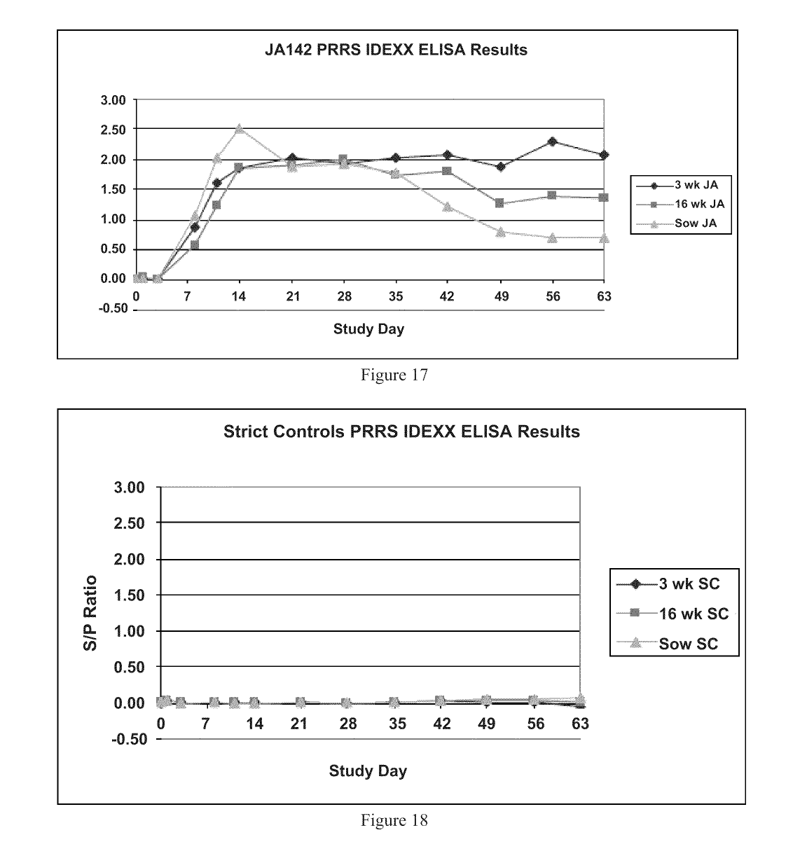

When comparing all of the animals challenged with JA142 by age group, the general trend for seroconversion was consistent. The decline in the S/P ratio after day 28 showed the most dissimilarity (see FIG. 17). The sows were quick to flush the antibodies from their system, whereas the 3 week piglets maintained the levels for the duration of the study. The Wilcoxon Two-Sample Test (p<0.05) data from days sampled show that the 3 week JA142 piglets have significantly different S/P ratios compared to the 16 week JA142 pigs on days 49-63. The 3 week JA142 piglets also have significantly different ratios than the JA142 sows on days 14, 56, and 63. Finally, the 16 week JA142 pigs' data is only significantly different from the JA142 sows on day 14.

All controls maintained undetectable levels of seroconversion in the serum for the duration of the study except for the minimal S/P value for strict control sows on day 63 (see FIG. 18). The statistical analysis revealed no days of significant differences except when you compare either the 3 week control piglets or the control 16 week pigs to the control sows on day 63 by the Wilcoxon Two-Sample Test (p<0.05).

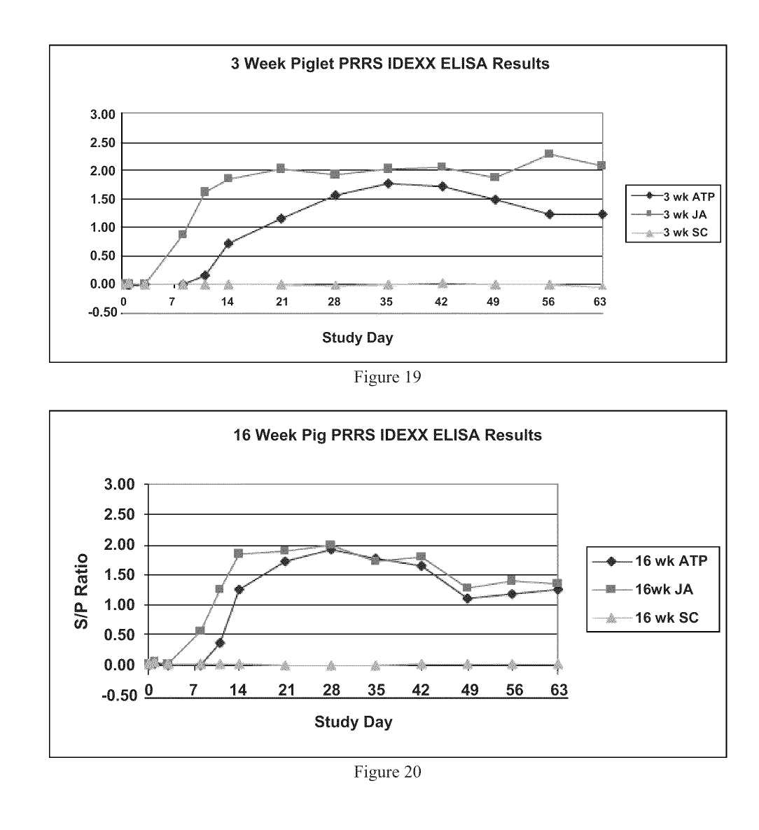

Seroconversion took place about 4 days sooner for the 3 week JA142 piglets than the 3 week ATP piglets (see FIG. 19). The 3 week JA142 piglets also achieved higher overall S/P ratios than the other two groups. The 3 week controls maintained undetectable levels of seroconversion for the duration of the study. The Wilcoxon Two-Sample Test (p<0.05) data from days sampled show that the 3 week ATP piglets achieved significantly different values from the 3 week JA142 piglets on days 8 through 21. The ATP piglets were also significantly different from the strict control piglets on all days except days 3 and 8. Finally, the 3 week JA142 piglets have significantly different S/P ratios than the strict control piglets on days 8 through 63.

Seroconversion took place about 4 days sooner for the 16 week JA142 piglets than the 16 week ATP piglets (see FIG. 20). The 16 week JA142 piglets also achieved higher overall S/P ratios than the other two groups. The 16 week controls maintained undetectable levels of seroconversion for the duration of the study. The Wilcoxon Two-Sample Test (p<0.05) data from days sampled show that the 16 week ATP pigs achieved significantly different values from the 16 week JA142 pigs on days 8 through 14, 49, and 63. When comparing to the 16 week strict controls, the 16 week ATP pigs have significantly different S/P values on days 11 through 63, and the 16 week JA142 pigs are significantly different on days 8 through 63.

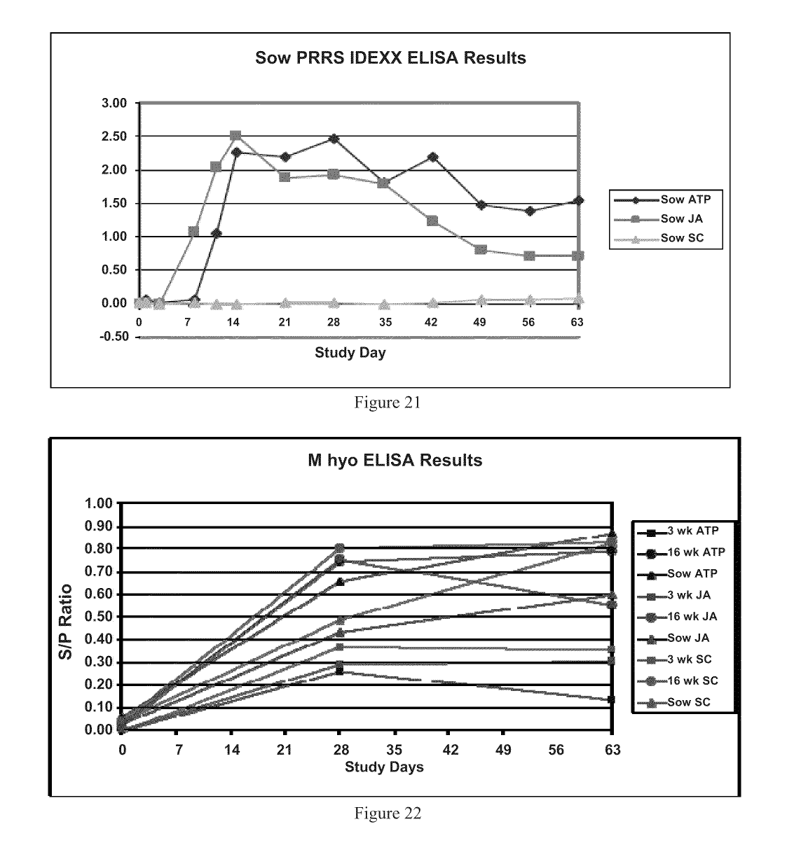

Seroconversion took place about 4 days sooner for the JA142 sows than the ATP sows (see FIG. 21). The JA142 sow also achieved slightly higher overall S/P ratios than the other two groups. The strict controls maintained undetectable levels of seroconversion until day 63 where there was a very minimal increase. The Wilcoxon Two-Sample Test (p<0.05) data from days sampled show that the ATP sows have significantly different S/P ratios than the JA142 sows on days 8, 11, and 42 through 63. The ATP sows and the JA142 sows are significantly different than the control sows on days 8 through 63.

### EXAMPLE 4

### Secondary Measures of Clinical Health

### Serology—IDEXX M. hyo ELISA (S/P Ratios) Assays

In FIG. 22, the three data points recorded for each group demonstrates a slight age effect with regard to seroconversion to the M. hyo vaccine, especially at day 28. Keep in mind that all pigs, including the strict controls (SC), received identical M. hyo injections. Statistical analysis shows no significance for the results comparing within the age groups. For example, the 3 week ATP piglets behaved the same as the 3 week JA142 piglets and the strict control 3 week piglets. The same is true for both the 16 week pigs and the sows. The Wilcoxon Two-Sample Test (p<0.05) data from days sampled show that when comparing all of the ATP challenged groups, the 3 week ATP piglets show significant differences in their S/P ratios when compared to both the 16 week ATP pigs and the ATP sows. However, comparisons between the 16 week ATP pigs and ATP sows show no difference. These observations are also true for the strict control groups. The JA142 groups showed no statistical differences.

### Gross Lung Lessions (VRI Vet Results)

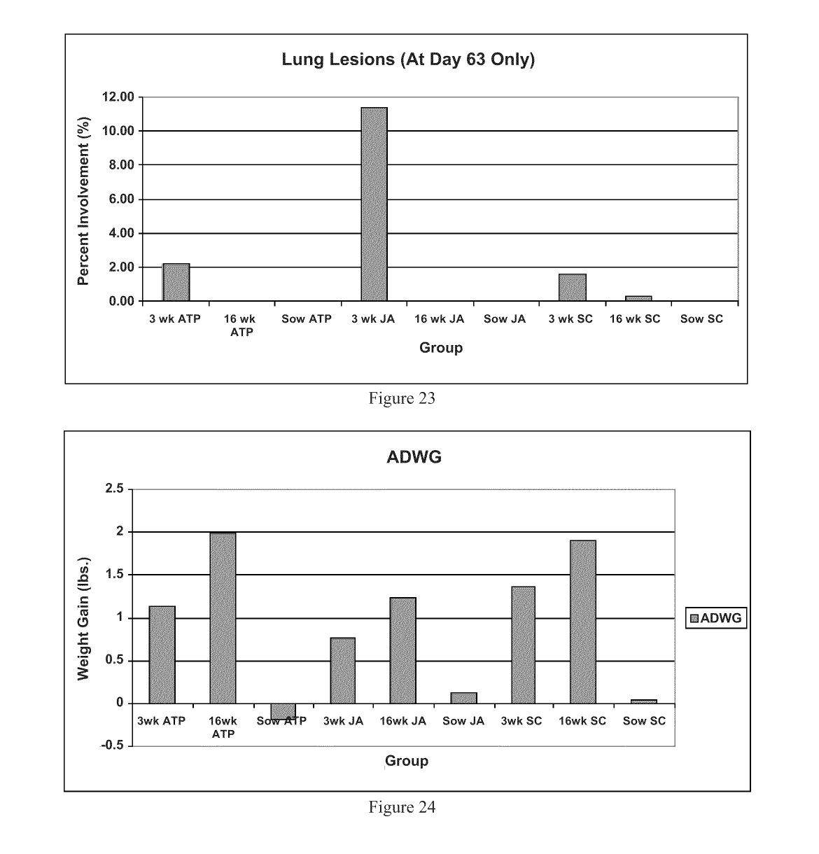

As expected at day 63 after challenge, negligible lung scores were reported. These scores do not indicate PRRS specific lesions, but a general, average percentage of consolidation in the lung (see FIG. 23). The Wilcoxon Two-Sample Test (p<0.05) data found statistical significance when comparing the 16 week ATP pigs and the 16 week JA142 pigs to the strict controls. Also, differences were found when comparing the 16 week ATP pigs and the ATP sows to the 3 week ATP piglets. The same was true for the JA142 piglets, pigs, and sows. Finally, the strict controls found statistically different lung score when comparing both the 3 week control piglets and the 16 week control pigs to the control sows.

Expected differences in Average Daily Weight Gain (ADWG) (see FIG. 24) were seen when comparing group means from all of the age groups (3 weeks vs. 16 weeks vs. sows.) Levene's Test of Homogeneity (p value <0.05) showed that within the 3 week age group on days 28 and 63, the ADWG is statistically significant when comparing the 3 week JA142 piglets to either the 3 week ATP piglets or the 3 week strict control piglets. The 16 weeks pigs only showed significance on day 28 when comparing the 16 week ATP pigs to the 16 week JA142 pigs. The sows showed no statistical differences.

### PRRS Immunohistochemistry Scores—Lungs (ISU)

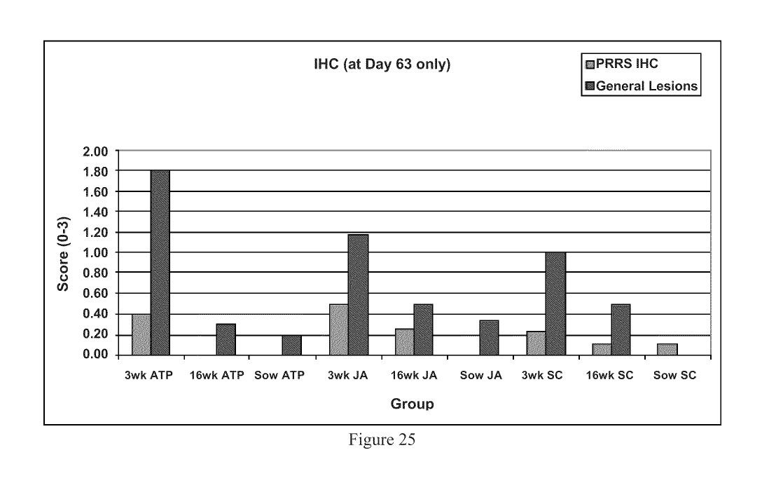

In FIG. 25 the general lesions bars show the non-specific lung score. The PRRS IHC scores reflect the PRRS specific staining of lung lesions. Scoring was as follows: if negative, the lung received a score of 0; if positive, the lung received a score of 1 to 3 depending of severity of lesion. The graph illustrates the lack of lesions remaining at the end of the study. For those existing lesions, even less were PRRS specific. Statistical analyses within each age group show no differences. The Wilcoxon Two-Sample Test (p<0.05) data found significant differences when comparing the 3 week ATP piglets to the 16 week ATP pigs and the ATP sows for the non-specific lesions only. The same was true for the JA142 groups. The only microscopic lesion comparison resulting in a significant difference was between the 3 week JA142 piglets and the JA142 sows.

### Clinical Scores

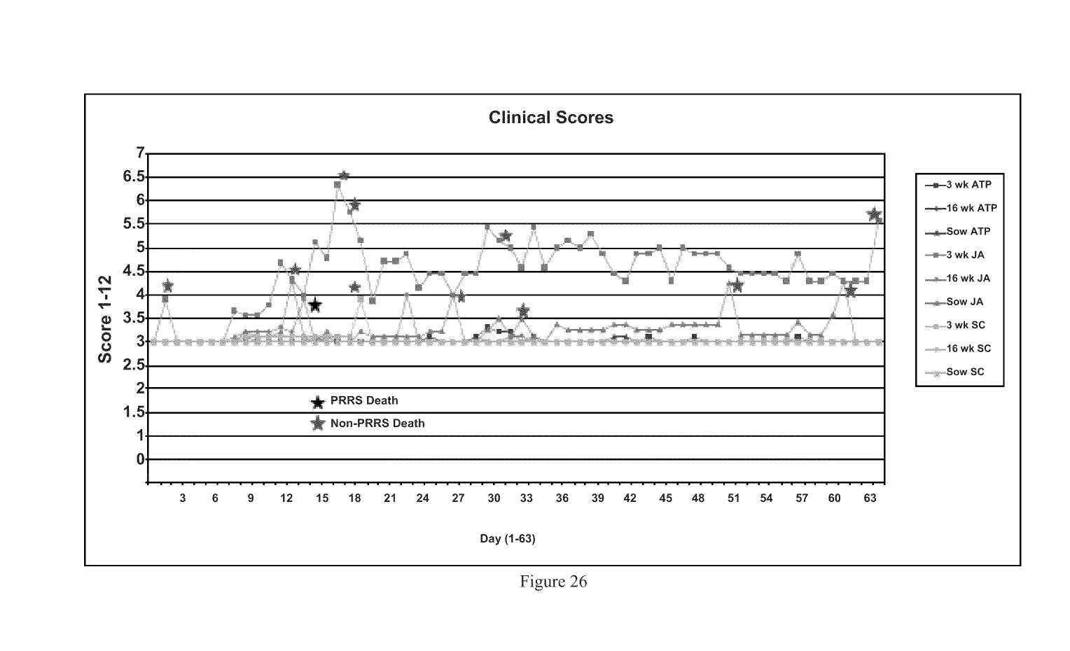

Due to death and the apparent clinical symptoms shown, group means were statistically significant from all other groups using the Wilcoxon Two-Sample Test (p<0.05) except for the following comparisons: ATP sow vs. control sows, 16 week ATP pigs vs. ATP sows, and 16 week control pigs vs. control sows (see FIG. 26). It should be noted that the death of a lame piglet in the 3 week control group skewed the control results.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the following claims.

### EXAMPLE 5

### Age-Dependent Resistance to Porcine Reproductive and Respiratory Syndrome Virus Replication in Swine

The present example shows that virulent PRRSV infection and disease were markedly more severe and prolonged in young piglets than in finishers or sows. Attenuated PRRSV in piglets also produced a prolonged viremia that was delayed and reduced in magnitude, and in finishers and sows, about half the animals showed no viremia. Despite marked differences in infection, antibody responses were observed in all animals irrespective of age, with older pigs tending to seroconvert sooner and achieve higher antibody levels than 3-week-old animals. Interferon γ (IFN γ) secreting peripheral blood mononuclear cells were more abundant in sows but not specifically increased by PRRSV infection in any age group, and interleukin-10 (IL-10) levels in blood were not correlated with PRRSV infection status.

This example leads to the conclusion that animal age, perhaps due to increased innate immune resistance, strongly influences the outcome of acute PRRSV infection, whereas an antibody response is triggered at a low threshold of infection that is independent of age. Prolonged infection was not due to IL-10-mediated immunosuppression, and PRRSV did not elicit a specific IFN γ response, especially in non-adult animals. Equivalent antibody responses were elicited in response to virulent and attenuated viruses, indicating that the antigenic mass necessary for an immune response is produced at a low level of infection, and is not predicted by viremic status. Thus, viral replication was occurring in lung or lymphoid tissues even though viremia was not always observed.

Experimental:

Ninety healthy, PRRS-negative pigs, consisting of 30 three-week-old weaned piglets, 30 16-20-week-old mixed sex finisher pigs, and 30 nonpregnant, third parity (±1) sows, were obtained from a PRRSV-free, genetically uniform, commercial source herd. Animals were confirmed PRRS-negative by HerdChek® PRRS 2XR ELISA (IDEXX Laboratories Inc., Westbrook, Me.) and given a Mycoplasma hyopneumoniae vaccine (Boehringer Ingelheim, St. Joseph, Mo.) on day 0 of the study. Animals were randomized by weight, within each age group, into 3 groups of 10 animals for infection with attenuated Ingelvac® PRRS ATP (Boehringer Ingelheim Vetmedica Inc., St. Joseph, Mo.), or virulent JA142 PRRSV (vaccine parental isolate, kindly provided by William Mengeling, National Animal Disease Center, Ames, Iowa) or received diluent only (Table 6). Viral isolates were diluted in Eagle's Minimal Essential Medium (EMEM) (SAFC Biosciences, Lenexa, Kans.) containing 4% fetal bovine serum (FBS) (SAFC Biosciences, Lenexa, Kans.) to approximately 3.0±0.5 log 10 TCID50/mL, as determined by titration on MA-104 cells [33]. Treatments were administered as a 1 mL intranasal inoculation and a 1 mL intramuscular injection. As the experiment was not a vaccine evaluation study, the Ingelvac® ATP virus was not taken from a vaccine formulation and the dose and route did not follow USDA-approved label recommendations. All animals were bled using Vacutainer® serum separation tubes (BD Biosciences, Franklin Lakes, N.J.). Serum samples were aliquoted and stored at −70° C. until use.

| Group | Age | Treatment | Sample size | Observations |
|---|---|---|---|---|
| 1 | Piglet | Ingelvac ® PRRS ATP | 10 | Clinical healtch, rectal |
| | | | | temperature daily. Blood and |
| | | | | serum weekly. Lung lesions |
| | | | | and tissue samples at |
| | | | | necropsy. |
| 2 | Finisher | Ingelvac ® PRRS ATP | 10 | Same as above |
| 3 | Sow | Ingelvac ® PRRS ATP | 10 | Same as above |
| 4 | Piglet | Virulent PRRSV JA 142 | 10 | Same as above |
| 5 | Finisher | Virulent PRRSV JA 142 | 10 | Same as above |
| 6 | Sow | Virulent PRRSV JA 142 | 10 | Same as above |
| 7 | Piglet | Culture media | 10 | Same as above |
| 8 | Finisher | Culture media | 10 | Same as above |
| 9 | Sow | Culture media | 10 | Same as above |

Viremia quantification: Ten-fold serial dilutions were carried out to a final dilution of 10⁻⁷ and four replicates of each dilution were plated on 96-well plates containing three-day-old MA-10⁴ cells. After incubation at 37° C. with 4.5% CO2 for eight days, wells were examined microscopically for cytopathic effect (CPE). Titer was determined as described (Reed, Am J. Hygiene, 1938, 27:493-497).

RNA extractions and qRT-PCR were performed as described [8]. Briefly, RNA was isolated by spin-column chromatography (QIAamp Viral RNA Mini-Kit, Qiagen Inc., Valencia, Calif.) and qRT-PCR was performed using a kit for quantitative detection of PRRSV in serum (Tetracore Inc., Gaithersburg, Md.). Results were reported as viral genome copies per mL.

Serological assays: Seroconversion was quantified as S/P ratios using the HerdChek® PRRS 2XR ELISA according to the manufacturer's instructions. Protein-specific ELISA was performed as described [19, 34]. Interleukin-10 levels were determined with a commercial ELISA kit (Biosource International, Camarillo, Calif.) following the manufacturer's instructions.

Cell-mediated immune assay Interferon γ secreting cells were enumerated in PBMC by ELISPOT as described (Xiao et al. 2004). PBMC were cultured at 5×10⁵ cells per well and were stimulated with PRRSV strain JA142 at 2×10⁵ TCID₅₀ per well.

Body weight: Each pig was weighed on days 0 and 63 of the study, using a calibrated, portable, electronic weigh-bar scale (Weigh-Tronix™ model 615XL, Weigh-Tronix Inc., Fairmont, Minn.). Three-week-old piglets and finishers were also weighed on day 28.

Clinical scores: Animals were observed daily for clinical condition. Individual scores for respiratory signs, coughing, and behavior were recorded on a scale from 1 (healthy) to 4 (most ill). A healthy pig received a daily score of 3, whereas a dead pig scored a 12. Animals that died prior to the end of the study were necropsied, evaluated for cause of death, and had samples collected for submission to the Iowa State University Diagnostic Lab for confirmation via pathological investigations.

Statistical analyses: Group mean data for TCID50, qRT-PCR, and IDEXX ELISA results was analyzed among age groups, and treatment type for statistical significance using the Kruskal-Wallis non-parametric ANOVA and individual comparisons were analyzed by the Wilcoxon two-sample t-test. Spearman coefficient correlation was used to compare the TCID50 and qRT-PCR parameters. A p value <0.05 was considered as statistically significant.

Results

Clinical signs and disease: Pigs in all age groups that were infected with virulent JA142 PRRSV showed clinical signs of PRRS, including coughing, which were slightly more severe in piglets (Table 7). Clinical signs were not evident in the attenuated ATP PRRSV-exposed and negative control animals. Ten pigs inoculated with virulent JA142 PRRSV died during the study. Causes of death varied, but only one, a finisher, was attributed to PRRS-related complications. One untreated piglet also died from a bacterial infection.

Piglets, which were the fastest growing group, showed a significantly reduced average daily weight gain (ADWG) at 28 and 63 days when infected with virulent PRRSV (Table 6). By contrast, inoculation with attenuated ATP PRRSV had no effect on ADWG in piglets. Twenty-week old pigs only showed reduced weight gain at 28 days when infected with virulent JA142 PRRSV (Table 7). There was no effect of PRRSV on weight gain in mature sows.

TABLE 7
Effect of PRRSV on clinical signs, clinical scores,
and weight gain in pigs of various ages.

Treatment Group

| | | |
|---|---|---|
| Control | ATP | JA 142 |

Clinical signs and scores

| | | | |
|---|---|---|---|
| Piglet | Normal | Normal | Mild cough, days 7-63 |
| | | | Range 3.0-6.3 (peak on day 16 |
| Finisher | Normal | Normal | Mild, sporadic cough |
| | | | Range3.0-4.0 (peak on day 22) |
| Sow | Normal | Normal | Mild cough, days 12-63 |
| | | | Range 3.0-4.3 (peak on day 12) |

Weight gain 0-28 d

| | | | |
|---|---|---|---|
| Piglet | 0.9 | 0.9 | 0.4* |
| Finisher | 2.1 | 2.3 | 0.9* |

Weight gain 28-63 d

| | | | |
|---|---|---|---|
| Piglet | 1.7 | 1.3 | 1.1* |
| Finisher | 1.8 | 1.8 | 2.0 |

Characteristics of Infection: Twenty-six of 30 animals in all age groups receiving virulent, JA142 PRRSV were viremic by day 1 and 100% were viremic on day 3 (Table 7). Viremia peaked on day 3 in finishers and sows with mean group titers of about 3.0 log 10 TCID₅₀/mL (FIG. 1). All animals in these groups cleared virus below the level of TCID₅₀ detection (≦10¹) by day 11 and, with one exception, remained negative to the end of the study. The exception, a sow, showed a low titer one time, on day 42. By contrast, viremia in piglets peaked on day 1 at a significantly higher titer of 4.5 log 10 TCID₅₀/mL. All piglets were viremic through 14 days of infection, and 6 of 7 were viremic at 21 days (Table 8). All piglets were negative at day 35.

The animals exposed to ATP PRRSV showed a substantially different pattern of viremia. The highest viremic load was observed in piglets, as was observed with virulent JA142, but virus was not detected until day 8, when 7 of 10 animals were positive. Two animals remained negative until day 21 (Table 8). Peak viremia, at 3.3 log 10 TCID₅₀/mL, occurred on day 21 and viral load declined slowly and variably. Seven of 10 piglets cleared the ATP PRRSV by day 42, but sporadic low level positives were observed for the duration of the study. By contrast, in growing finishers and sows, only 3 to 4 of 10 animals had detectable levels of viremia on day 3 (Table 8). Peak mean group titers were reached on day 3 in finishers (0.73 log 10 TCID₅₀/mL) and on day 8 in sows (1.49 log 10 TCID₅₀/mL), but variation in viremia among animals was substantial. Five finishers and four sows did not show viremia during the entire study. Viremia was not observed in finishers or sows after 11 days except for 2 finishers that were viremic on day 21. All 30 non-challenged control animals remained PRRSV-negative for the duration of the 63 day study.

TABLE 8
Proportion of PRRSV-positive animals by viral isolation on MA104 cells.

| | | Proportion of Viremic animals at the indicated days of infection* | | | | | | | | | | | | |
|---|---|---|---|---|---|---|---|---|---|---|---|---|---|---|
| Treatment | Pig age | 0 | 1 | 3 | 8 | 11 | 14 | 21 | 28 | 35 | 42 | 49 | 56 | 63 |
| ATP | Piglet | 0/10 | 0/10 | 0/10 | 7/10 | 8/10 | 8/10 | 9/10 | 8/10 | 8/10 | 3/10 | 3/10 | 2/10 | 0/10 |
| | Finisher | 0/10 | 0/10 | 3/10 | 2/10 | 1/10 | 0/10 | 2/10 | 0/10 | 0/10 | 0/10 | 0/10 | 0/10 | 0/10 |
| | Adult | 0/10 | 0/10 | 4/10 | 5/10 | 4/10 | 0/10 | 0/10 | 0/10 | 0/10 | 0/10 | 0/10 | 0/10 | 0/10 |
| JA 142 | Piglet | 0/10 | 9/9 | 9/9 | 9/9 | 9/9 | 9/9 | 6/7 | 4/7 | 0/6 | 0/6 | 0/6 | 0/6 | 0/6 |
| | Finisher | 0/10 | 8/10 | 10/10 | 4/9 | 0/9 | 0/9 | 0/9 | 0/8 | 0/8 | 0/8 | 0/8 | 0/8 | 0/8 |
| | Adult | 0/10 | 9/10 | 10/10 | 7/10 | 0/10 | 0/9 | 0/9 | 0/8 | 0/8 | 0/8 | 0/8 | 0/7 | 0/6 |
| None | Piglet | 0/10 | 0/10 | 0/10 | 0/10 | 0/10 | 0/10 | 0/10 | 0/10 | 0/10 | 0/10 | 0/10 | 0/10 | 0/10 |
| | Finisher | 0/10 | 0/10 | 0/10 | 0/10 | 0/10 | 0/10 | 0/10 | 0/10 | 0/10 | 0/10 | 0/10 | 0/10 | 0/10 |
| | Adult | 0/10 | 0/10 | 0/10 | 0/10 | 0/10 | 0/10 | 0/10 | 0/10 | 0/10 | 0/10 | 0/10 | 0/10 | 0/10 |

*All groups with at least one positive animal are shown in bold.

Viremia also was determined by qRT-PCR; the results were significantly correlated with viral isolation on MA-104 cells. Spearman correlation coefficients ranged from 0.6 to 0.8 and all were significant (p<0.05). The qRT-PCR findings confirmed the TCID₅₀/mL results obtained by growth on MA-104 cells, indicating that both the virulent and attenuated strains grew equivalently in cell culture. In young piglets infected with virulent JA142, viremia was high on day 1 and remained high until day 28, after which it declined substantially. In finishers and sows exposed to JA142, viremia was high from day 1 to day 14, then declined dramatically. Attenuated ATP PRRSV elicited similar kinetics in young, growing and mature pigs; a gradual increase until day 21 followed by a gradual decline in piglets, and brief, low-level viremia in finishers and sows.

Except for day 1, when the copies/mL of viral RNA were significantly different among all three age groups, JA142-infected piglets had significantly higher levels of viremia than both finishers and sows, which were equivalent (ttest, p<0.05). ATP PRRSV-treated piglets also had significantly higher levels of viremia by qRT-PCR than finishers or sows on days 14 through 42, day 56, and day 63.

Characteristics of the immune response: All pigs showed the same serological response to acute infection with virulent JA142 regardless of age. As shown in FIG. 15, all group means were positive at day 8 and peaked at 14 to 21 days, as determined by HerdChek PRRS 2XR ELISA. All groups maintained a positive sample-to-positive (S/P) ratio for the duration of the study (FIG. 15). Sows showed a substantial decline in S/P ratio after 35 days, while piglets showed the highest average S/P ratio from 49 to 63 days after infection.

Animals inoculated with attenuated ATP PRRSV seroconverted at 10 to 14 days, with sows responding on average more rapidly than finishers and piglets. The response of sows to attenuated ATP PRRSV peaked at 14 days, whereas finishers showed increased mean S/P ratios until day 28 and piglets showed a gradual increase in S/P ratio up to 35 days. Thereafter, animals appeared to equalize and then maintain comparable S/P ratios for the duration of the study.

In FIG. 15, the antibody response of piglets to attenuated PRRSV appeared to be delayed. To further investigate this possibility, we examined antibody responses to two specific viral polypeptides. The kinetics of anti-N responses were essentially the same among all ages as determined by HerdChek® PRRS 2XR ELISA, including the declining response of sows infected with virulent PRRSV, and the increasing level of anti-N antibodies from days 49 to 63 in piglets infected with virulent PRRSV (FIG. 27A). The response of piglets to attenuated PRRSV was not significantly different from that of other age groups. Antibody responses to a second viral antigen, an antigenic polypeptide fragment of nonstructural protein 2 (nsp2Hp), showed another pattern of reactivity. Here, virulent JA142.

PRRSV elicited antibody responses that peaked at 21 to 28 days in all three age groups, then declined slightly in piglets and finishers, and substantially in sows (FIG. 27B). Attenuated ATP PRRSV elicited lower levels of antinsp2Hp that peaked at 28 days and were maintained for the duration of the study, or declined slightly in piglets (FIG. 27B). Anti-nsp2Hp responses were lowest in sows inoculated with attenuated PRRSV (FIG. 27B), but the same group showed a strong response in HerdChek® PRRS 2XR ELISA (FIG. 15). Thus, pigs of all ages mount a humoral immune response to both virulent and attenuated PRRSV, though its appearance tends to be more rapid in response to virulent virus exposure.

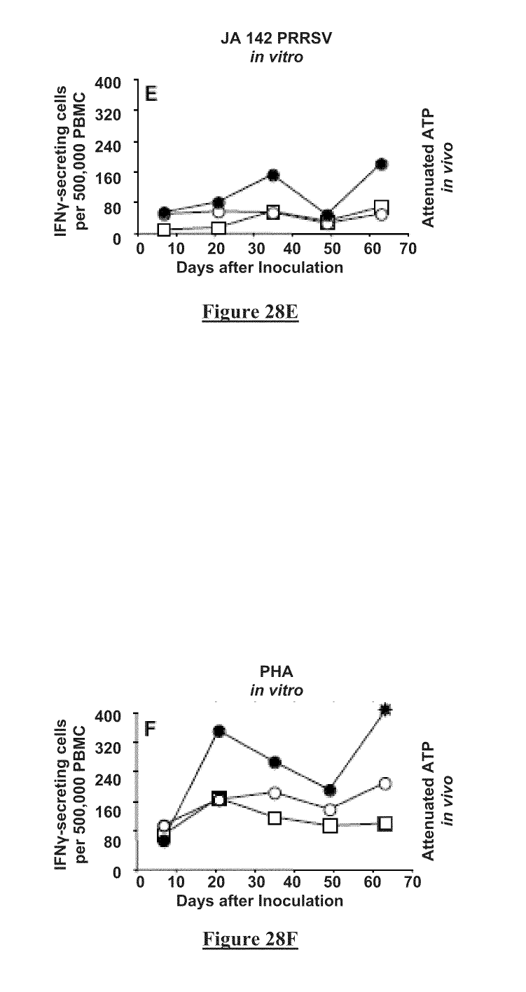

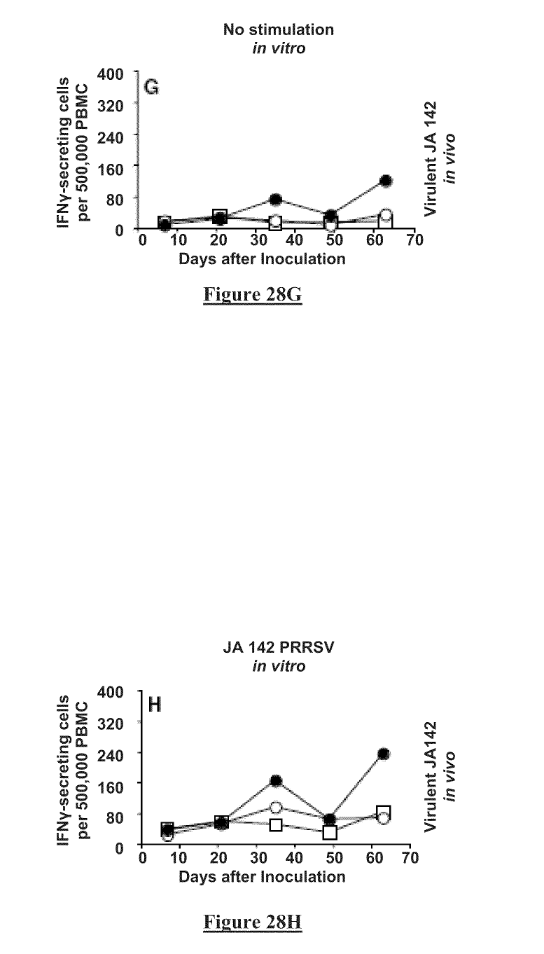

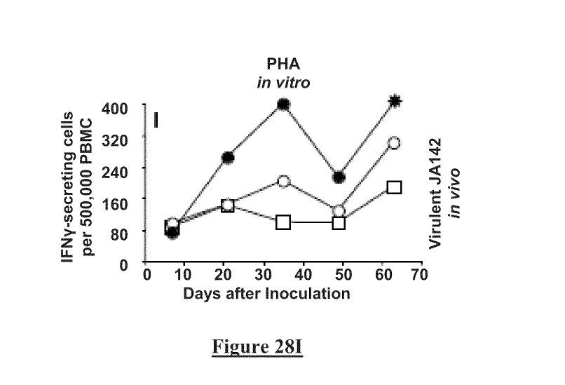

Cell-mediated immune responses were examined by IFNγ ELISPOT for evidence that they could explain anti-PRRSV immunity that was not accounted for by antibody responses. Uninfected healthy piglets and finishers showed very low levels of constitutive IFNγ secretion in peripheral blood mononuclear cells (PBMC) alone or after in vitro stimulation with virulent PRRSV, whereas mitogenic stimulation increased the frequency of secreting cells (FIG. 28A-C, open circles and open squares). The outcome was similar in piglets and finishers inoculated with attenuated PRRSV, although in vitro stimulation with virulent PRRSV increased secreting cell numbers (FIG. 28D,E). PBMC from piglets and finishers infected with virulent PRRSV showed the highest levels of IFNγ secretion under all culture conditions, although there was no consistent change over time (FIG. 28 G-I). Sows under all conditions of in vivo virus exposure and in vitro culture had higher frequencies of IFNγ secreting cells than did piglets and finishers (p<10⁻⁶, χ² test). In other respects the trends were the same as in piglets and finishers. Thus, cell-mediated immunity, based on IFNγ secreting cell responses, showed age-dependent variation that was not observed in anti-PRRSV antibody responses.

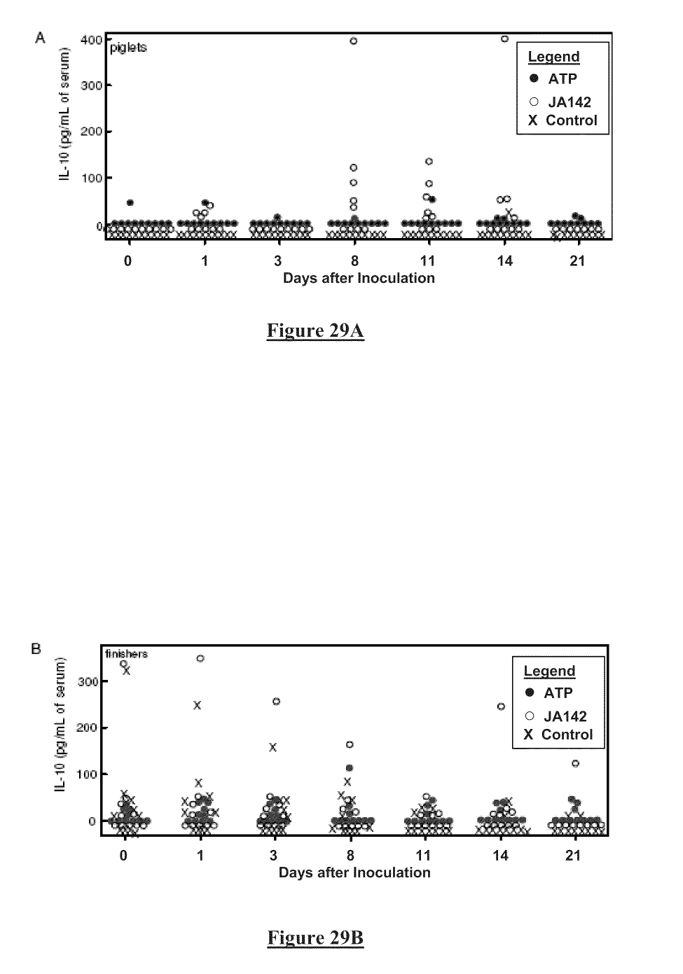

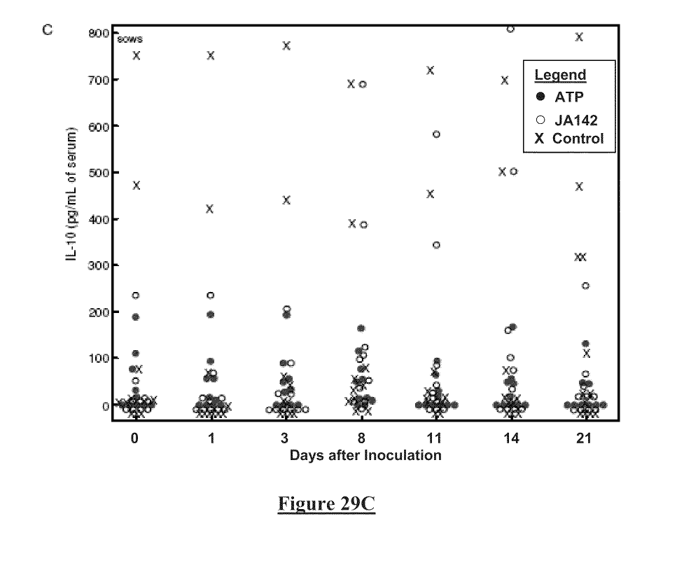

The level and duration of viremia were significantly greater in piglets than in finishers and sows. Therefore, IL-10 levels were determined in serum since it has been implicated in delayed immune responses to PRRSV infection. In piglets infected with virulent PRRSV, IL-10 levels were significantly increased in serum at 8-14 days of infection (FIG. 29A, p<0.05). There was no difference between pigs inoculated with attenuated virus or controls. IL-10 levels were more variable in finishers and sows, and there was no difference due to treatment (FIG. 29 B, C). In contrast to piglets, approximately half of the finishers and sows before virus exposure had measurable levels of IL-10 that were maintained throughout the study. The results indicate that increased IL-10 levels in serum are associated with age, and that in piglets, increased IL-10 levels are related to viral pathogenesis but do not modulate antiviral immunity.

The data presented here show that the consequences of PRRSV infection are highly dependent on pig age. Viral growth is most extensive in piglets. For both virulent and attenuated PRRSV, peak viremia and duration were substantially greater in piglets. Finishers and sows showed the same pattern of low level viremia for virulent viral infection that resolved within 2 weeks, and approximately 50% of finishers and sows inoculated with attenuated PRRSV showed no viremia. The prolonged period of viremia commonly associated with PRRSV infection is based on studies in young pigs. The finding that viremia is substantially reduced in growing and adult pigs is novel and indicates that the mechanisms of PRRSV resistance are developmentally regulated.

### REFERENCES

The following references, to the extent that they provide exemplary procedural or other details supplementary to those set forth herein, are specifically incorporated herein by reference.
Klinge, K. L, Roof, M. B., Vaughn, E. M., and Murtaugh, M. P., “PRRSV Replication and Subsequent Immune Responses in Swine of Various Ages”, Abstract of Poster No. 56, International Porcine Reproductive and Respiratory Syndrome (PRRS) Symposium, PRRS and PRRSV—Related Diseases: Prevention and Control Strategies, Chicago, Ill., Nov. 30-Dec. 1, 2007.
Klinge, K. L, Vaughn, E. M., Roof, M. B., Bautista, E. M. and Murtaugh, M. P., “Age-dependent resistance to Porcine reproductive and respiratory syndrome virus replication in swine”, Virology Journal 2009, 6:177.

What is claimed is:

1. A method of treating or reducing the severity of or incidence of porcine reproductive and respiratory syndrome virus (PRRSV) infection comprising administering a therapeutic amount of a PRRSV antigen to a piglet of about three weeks or younger.

2. The method of claim 1, wherein said PRRSV antigen is a modified live PRRS virus.

3. The method of claim 1, wherein said PRRSV antigen is Ingelvac® ATP.

4. The method of claim 1, wherein the PRRSV antigen is administered nasally.

5. The method of claim 1, wherein the PRRSV antigen is administered in a single dose.

6. A method of preventing porcine reproductive and respiratory syndrome virus (PRRSV) infection comprising administering a therapeutic amount of a PRRSV antigen to a piglet of about three weeks or younger.

7. The method of claim 6, wherein said PRRSV antigen is a modified live PRRS virus.

8. The method of claim 6, wherein said PRRSV antigen is Ingelvac® ATP.

9. The method of claim 6, wherein the PRRSV antigen is administered nasally.

10. The method of claim 4, wherein the PRRSV antigen is administered in a single dose.

11. A method of treating or reducing the severity of or incidence of porcine reproductive and respiratory syndrome virus (PRRSV) infection comprising administering a therapeutic amount of a PRRSV antigen to a pig about sixteen weeks old.

12. The method of claim 11, wherein said PRRSV antigen is a modified live PRRS virus.

13. The method of claim 11, wherein said PRRSV antigen is Ingelvac® ATP.

14. The method of claim 11, wherein the PRRSV antigen is administered nasally.

15. The method of claim 11, wherein the PRRSV antigen is administered in a single dose.

16. The method of claim 14, wherein the PRRSV antigen is administered in a single dose.

17. A method of preventing porcine reproductive and respiratory syndrome virus (PRRSV) infection comprising administering a therapeutic amount of a PRRSV antigen to a pig about sixteen weeks old.

18. The method of claim 17, wherein said PRRSV antigen is a modified live PRRS virus.

19. The method of claim 17, wherein said PRRSV antigen is Ingelvac® ATP.

20. The method of claim 17, wherein the PRRSV antigen is administered nasally.

21. A method of determining the proper timing and dosage for vaccination of a pig against PRRSV, said method comprising the steps of:
a) determining the age of the pig;
b) determining the health status of the pig;
c) determining the innate and active immunity for pigs of similar age and health status by comparing the age and health status with a standard for pigs of similar age and health status; and
d) determining the proper timing and dosage for vaccination by modifying a standard dosage for a pig of similar age and health status based on the age, health status, innate immunity levels and active immunity levels of the pig.
